(19) United States
(12) Patent Application Publication
(10) Pub. No.: US 2003/0118563 A1
(43) Pub. Date: Jun. 26, 2003

(54) Materials and methods for repair of tissue

(76) Inventor: Marvin P. Loeb, Huntington Beach, CA (US)

Correspondence Address: OLSON & HIERL, LTD., 36th Floor, 20 North Wacker Drive, Chicago, IL, 60606, US

(21) Appl. No.: 10/154,259

(22) Filed: May 23, 2002

Related U.S. Application Data
(63) Continuation-in-part of application No. 09/406,257, filed on Sep. 23, 1999, now abandoned.

(51) Int. Cl. A61K 48/00; C12N 5/08; A61N 1/30

(52) U.S. Cl. 424/93.21; 435/372; 604/20

(57) ABSTRACT

Stem cells, bone marrow, or bone marrow enriched with stem cells, is introduced into a host's tissue deficient in blood flow or deficient in a cell type, preferably by creating a trap or pocket within the tissue for confining introduced material. The trap or pocket can be created using mechanical and light energy, or like expedients.

### CROSS-REFERENCE TO RELATED APPLICATION

This application is a continuation-in-part of co-pending U.S. patent application Ser. No. 09/406,257, filed on Sep. 23, 1999.

### TECHNICAL FIELD OF THE INVENTION

The present invention relates to induction of angiogenesis in and the cellular repair of mammalian tissue. The material induces angiogenesis and supplies new cells in the repair of tissue in a physiologically compatible manner. Revascularization and cellular repair can be achieved in heart tissue, wounds, surgical incisions, the brain, the spinal cord, muscles of the extremities and other tissues deficient in blood flow, including the male scalp, as well in tissues in need of additional viable cells.

### BACKGROUND OF THE INVENTION

Coronary Heart Disease and TMLR—Coronary Heart disease is prevalent in modern society. Reduced blood supply to the heart, due to blockages in one or more of the coronary arteries, is the most common cause of heart attacks and death from heart disease. Currently, surgical intervention using coronary artery bypass graft surgery and/or coronary balloon angioplasty is the most common procedure to treat this condition. Normally, a person can only undergo coronary bypass surgery twice, since the risks will begin to outweigh the benefits after that point. Thus, in the past, a patient who has already had two coronary bypass surgeries was left without recourse. Other patients have failed repeated coronary balloon angioplasties, or are not suitable candidates for coronary bypass surgery or coronary balloon angioplasty. These persons likewise are left without treatment options.

Early attempts to create direct blood supply to the myocardium of mammals through channels from the heart chamber, as in lizards and other reptiles, involved producing tiny channels or passages in mammalian and human hearts with needles or pre-heated wires. These methods met with limited success since the channels soon healed over entirely and failed to continue to enhance the blood supply. Early attempts were made to graft a blood vessel from the aorta directly into the heart muscle to provide an internal source of blood. While some benefits were seen, the surgery was technically demanding and the procedure was eclipsed by the introduction of coronary artery bypass graft surgery.

To overcome these problems, transmyocardial laser revascularization (TMLR) has been attempted using a pulsed CO₂ laser to make the channels; Mirhoseini et al., “Revascularization of the Heart by Laser”, J Microsurg 2:253 (June, 1981). The laser forms each channel by vaporizing a passageway completely through the wall of the heart, enabling blood from the heart chamber to perfuse the heart muscle. The relatively clean channel formed by the laser energy prevents the channel from quickly healing over, and the channel either closes by clotting at the heart's outer surface, due to exposure to air or application of manual pressure. In some cases, a suture is required to close the channel. However, if bleeding cannot be stopped, or if bleeding resumes at a later time, after the patient is no longer in surgery, the patient may require emergency surgery or may die.

The patents report immediate reduction in angina pain as a result of blood flow from the chamber and, probably, temporary deactivation of nerves in the area of the channels. However, while most, if not all of the laser created channels close over a period of one or more months, the reduction in angina pain produced by TMLR increases over a period of six months and is stable for at least an additional six months. In animal studies, it was found that extensive angiogenesis was seen in the area surrounding the channels, which is believed to compensate for the eventual closing of the channels and produce the increasing benefit over six months or longer.

Since the body stores only small amounts of angiogenic growth factors in the heart, it is obvious that supplementing the body's supply of natural (endogenous) growth factors with growth factors produced by recombinant technology, or infecting the myocardium with genes able to cause myocardial cells to express angiogenic growth factors, could yield greater angiogenesis and thus greater therapeutic benefits.

An giogenesis—Angiogenesis is the fundamental process by which mammalian systems form new capillary blood vessels in normal growth and in response to injury. Normal angiogenesis is tightly regulated, and excessive angiogenesis has been implicated in many disease states, including cancer. Arteriogenesis entails the formation of arterioles or neo-arteries which, unlike capillaries, are muscularized vessels providing greater blood flow. Specific angiogenic growth factors and other substances have been identified in the art, such as vascular endothelial growth factor or VEGF, fibroblast growth factor or FGF, and agents which cause blood vessels to mature, such as angiopoetin. (See for example Folkman et al., J. Biochemistry 267(16):10931-10934 (1992); Thomas, J. Biochemistry 271(2):603-606 (1996).

Initial work in the area of angiogenesis revolved around the discovery and characterization of angiogenic agents. For example, Abraham et al., “Nucleotide Sequence of a Bovine Clone Encoding the Angiogenic Protein, Basic Fibroblast Growth Factor”, Science, 233:545-548 (1986) taught the nucleotide sequence of acidic FGF (aFGF or FGF-1), and the structures of acidic FGF and basic FGF (bFGF or FGF-2).

Recently it has been shown that the administration of purified human FGF-I was able to induce neoangiogenesis in ischemic myocardium, after injection into the heart muscle concurrent with internal mammary artery (IMA)/left anterior descending coronary artery (LAD) anastomosis surgery. Schumacher et al., “Induction of Neoangiogenesis in Ischemic Myocardium by Human Growth Factors” Circulation, 97: 645-650 (1998).

Gene Therapy—With the identification and characterization of various angiogenic agents, it was possible to pursue direct molecular intervention in vivo of the processes of neovascularization. Gene therapy has been a long desired goal of biomedical science, but effective introduction of genes causing the expression of VEGF or FGF into cells of the myocardium takes lengthy exposure or “residence” time, which is not practical in a beating heart. Inserting an angiogenic gene into the genome of a replication deficient virus, such as Adenovirus, which retains its ability to infect cells but is unable to replicate, was proposed to overcome this problem. Berlener, “Development of Adenovirus Vectors for the Expression of Heterologous Genes”, Biotechniques 6:616-629 (1988) was one of the earliest reports on the use of such viruses for gene transfer. “A Therapeutic Window for In Vivo Adenoviral-Mediated Gene Transfer”, Circulation 90(4), part 2:I-516, Abstract #2778 (1994), illustrates the various viral concentrations beyond which efficiency is not increased, using a rat carotid artery system.

Continued research on gene therapy and angiogenic factors have yielded information about coordinated action of various factors, for example, Suri et al., “Increased Vascularization in Mice Overexpressing Angiopoetin-1”, Science 282:468-471 (1998), showed that angiopoetin-1 is necessary to mature and maintain new vessels initially created by introduction of VEGF or aFGF. This work demonstrates that additional substances, such as angiopoietin-1, can be used to maintain the integrity of the newly created vessels for a long term effect.

Recently, injection of 4000 μg of gene (naked DNA) for VEGF into leg muscles of humans with peripheral atherosclerosis and limb ischemia was shown to benefit more than half the subjects. However, a significant percentage did not respond to the therapy. See Baumgartner, I. et al, “Constitutive Expression of ph VEGF₁₆₅ After Intramuscular Gene Transfer Promotes Collateral Vessel Development in Patients with Critical Limb Ischemia Circulation” 1114-1123, Mar. 31, 1998.

Coronary Heart Disease, Angiogenesis and Infusion—With greater understanding about angiogenic growth factors and genes expressing the same, collectively “angiogenic agents”, and their potential to induce neovascularization, infusion of such angiogenic agents into one or more coronary arteries was attempted to increase blood supply to the heart. However, a deficiency of this route of administration is that the angiogenic agent passes quickly through the artery into the general circulation after only one heartbeat.

The use of angiogenic agents and their potential for treating heart disease were discussed by Goldsmith, “Tomorrow's Gene Therapy Suggests Plenteous, Potent Cardiac Vessels”, JAMA 268(23):3285-3286 (1992). In this article, Goldsmith discusses work by Jeffrey Leiden & Elian Barr (U. of Chicago), including naked DNA injection into cardiac and skeletal muscle and the use of an adenovirus (replication sequences deleted) vector containing an angiogenic gene which was injected into a coronary artery, infecting the entire artery.

Further work by Barr et al., “Efficient Catheter-Mediated Gene Transfer into the Heart Using Replication-Defective Adenovirus”, Gene Therapy 1:51-58 (1994), showed that five days after intra-coronary artery infusion an angiogenic gene inserted into the plasmid of a replication deficient adenovirus, the virus was detected in the brain, lungs, liver, kidneys and testes. This was after a single infusion into a coronary artery of 2×10⁹−1×10¹⁰ p.f.u. of adenovirus-linked gene. Thus, infusion of adenovirus-linked angiogenic genes into a coronary artery resulted in the undesirable result of disseminating angiogenic capable genes systemically. This could enable an occult tumor to grow by extending its blood vessel system. Also, the body's immune system attacks and kills the cells invaded by the virus, limiting the duration of action to days or weeks.

Angiogenesis by Intramuscular Injection—Attempts to directly inject angiogenic agents directly into the muscle of the heart, while attractive, have had various technical difficulties that reduces the overall efficacy of gene therapy. Lin et al., “Expression of Recombinant Genes in Myocardium after Direct Injection of DNA”, Circulation 82: 2217-2222, (1990), showed the feasibility of gene transfer into the cells of the myocardium by direct injection of naked DNA. However, when therapeutic agents, in a liquid medium, are injected into the wall of a beating heart, much of the liquid is expelled by contraction of the heart muscle on its next compression.

Studies of the specific transformation of heart muscle cells was greatly advanced by the work of Barr et al., “Systemic Delivery of Recombinant Proteins by Genetically Modified Myoblasts”, Science 254:1507-1509 (1991) demonstrated that skeletal muscle cells of a host could be infected by a virus linked to an angiogenic gene and injected into the myocardium. This was useful since myocytes cannot be cultured in-vitro. However, it was found that injection of these cells into cardiac muscle, resulted in an inflammatory response and fibrous formations. Only transitory gene expression was seen, due to the host's immune system killing the cells infected by the “foreign” viral vector.

French et al., “Feasibility and Limitations of Direct In Vivo Gene Transfer into Porcine Myocardium Using Replication-Deficient Adenoviral Vectors”, Circulation 90(4), part 2:I-517, Abstract #2785 (1994), observed a much higher efficiency of transformation (140,000 times higher) using a viral vector linked to a gene, versus the gene alone. Angiogenic transformation using viral vector/gene injection rarely produced angiogenesis more than 5 mm from the injection site. Again, due to an immune response, the duration of gene activity was limited.

Losordo et al., “Gene Therapy for Myocardial Angiogenesis”, Circulation 98:2800-2804 (1998), describes initial clinical results with direct myocardial injection of ph VEGF₁₆₅ as sole therapy for myocardial ischemia in persons who had failed conventional therapy, and suffered from angina. Naked plasmid DNA encoding for VEGF was injected directly into the ischemic myocardium (anterolateral left ventricular free wall) via a mini left anterior thoracotomy (125 μg in 4 aliquots of 2.0 ml each). After about 60 days post-operation, the patients appeared to benefit from the treatment.

In an oral presentation at the angiogenesis and direct myocardial revascularization meeting in Washington, D.C. on Jul. 10, 1999, Burkhoff, D. (No printed abstract) described how bovine (cow) long bones were ground, acid demineralized, purified, chromatographically (HPLC) separated and formulated into a mixture, called Bone Matrix, which was injected into the yolks of Japanese quail eggs and produced significant angiogenesis and evidence of arteriogenesis. When the Bone Matrix was injected into rats, however, an inflammatory (cross species) response was seen, which lasted six weeks, limiting its duration of action.

Several U.S. patents are related to gene therapy, viral vectors, and in particular angiogenic agents, and the TMLR procedure, including U.S. Pat. Nos. 5,849,997 to (Grosveld et al.); 5,849,718 (Grosveld); 5,849,572 (Glorioso et al.); 5,846,947 (Behr et al.); 5,661,133 (Leiden et al.); 5,837,511 (Crystal et al.); 5,792,453 (Hammond et al.); 5,328,470 (Nabel et al.); 5,698,531 (Nabel et al.); 5,707,969 (Nabel et al.); 5,840,059 (March et al.,); 5,389,096 (Aita et al.,); and 5,554,152 (Aita et al.).

While an angiogenic growth factor, a gene coding for a growth factor, or such a gene incorporated in a vector, in a liquid form, may be injected into an arrested heart with a simple syringe, in the case of a beating heart, much of the angiogenic agent would be expelled on its next contraction. As a result, creating a space within the heart muscle, in which the angiogenic agent could reside for a sufficient time to assure its absorption into cells of the heart wall would be desirable.

TMLR and Angiogenic Agent Therapy—Recently, Sayeed-Shah et al., “Complete Reversal of Ischemic Wall Motion Abnormalities by Combined Use of Gene Therapy With Transmyocardial Laser Revascularization”, J. Thorac. Cardiovasc. Surg. 116(5):763-9 (1998) describe the injection of VEGF genes along with TMLR. The combined TMLR/gene therapy was able to normalize heart wall motion in animals in which a coronary artery was artificially constricted, whereas heart wall motion was not normalized in similarly ischemic animals by the injection of the same gene or TMLR alone.

The prior art CO₂ laser uses several mirrors mounted on an articulating arm to reflect its light energy through a surgically created opening in the patient's chest, since CO₂ laser energy cannot be transmitted efficiently through optical fibers. Laser energy transmitted through flexible optical fibers, through a puncture between the ribs or percutaneously through a major artery leading to the heart chamber, could eliminate the need to create an opening in the patient's chest in order to perform the TMLR procedure.

Further, the use of lasers whose energy can be transmitted through optical fibers, such as argon-ion, have also been proposed for performing TMLR through a percutaneously inserted catheter from the inside of the heart chamber, Lee et al., “Effects of Laser Irradiation Delivered by Flexible Fiberoptic System on the Left Ventricular internal Myocardium”, Am Heart J., 63, Pg 587-590, September, 1983.

However, if argon-ion laser energy is applied to form the channel completely through the heart wall, the optical fiber must be present in the heart wall for a period of time longer than diastole, when the heart's electrical activity is minimal and the heart is momentarily at rest, since such lasers are of significantly less power than the CO₂ laser used in TMLR. If the procedure cannot be completed during diastole, within approximately 0.6 seconds (at a heart rate of 60 beats per minute), between heartbeats when the heart's electrical activity is minimal, a life threatening arrhythmia may result, and mechanical damage may occur to the heart muscle during its compression, which is impeded by the presence of the fiber.

The present invention avoids, or at least minimizes, the problems of the prior art methods of administration of an angiogenic agent and accomplishes the desired angiogenesis and arteriogenesis in a physiologically compatible manner. In addition, the present invention facilitates repair of mammalian tissues.

### SUMMARY OF THE INVENTION

An angiogenesis inducing material is introduced into a channel or space created in living tissue, e.g., into a muscle such as the wall of a beating heart. A preferred material for this purpose is a patient's own bone marrow. The bone marrow contains presently identified growth factors, genes and associated agents (promoters, enhancers, etc.), as well as a wealth of presently unidentified growth factors, promoters, enhancers and cytokines (signaling agents). Vitality of tissue which contains dead or damaged cells is restored or the tissue is repaired by the patient's own bone marrow which provides a supply of stem cells which, in certain instances, the body's existing signalling processes can transform into the type of cell the tissue requires, or by other bone marrow. Stem cells and bone marrow from a donor physiologically compatible with the patient can be utilized for the present purpose as well.

In practicing a preferred aspect of the present invention, an aliquot of autologus (host) bone marrow is withdrawn from the host's hip, spine or long bones using a syringe in a manner known in the art. The bone marrow may, if desired, be passed through one or more screens of decreasing pore size and centrifuged to remove fat and red cells. The residual material, referred to as buffy coat, containing stem cells, white blood cells, platelets and intercellular bone marrow material, may be suspended in phosphate buffered saline (BS), tissue culture medium, (preferably serum free tissue culture medium), or other physically compatible liquid as is known in the art. One method for preparation of bone marrow for transplant purposes is described by Thomas, E. D. et al. “Technique for Human Marrow Grafting”, Blood, 36(4):507-515 (October 1970).

Embryonic stem cells as well as hematopoietic stem cells are suitable for present purposes. Stem cells collected from peripheral blood of the host, by means known in the art, may be added to enrich the bone marrow suspension. However, it is also contemplated that stem cells, which could be thixotropic, may be administered per se, i.e., not as part of the bone marrow suspension, especially where the stem cells were cultured in vitro for administration to a patient. Colloids or other agents, as known in the art, may be added to the stabilize the suspension of the bone marrow in the liquid for subsequent reinjected into the patient, if desired.

Devices and methods which can be used for in vivo injection of a therapeutically effective amount of the bone marrow suspension or whole bone marrow, are described in U.S. Pat. No. 4,788,975; U.S. Pat. No. 5,913,853; U.S. Pat. No. 5,984,915; U.S. Pat. No. 5,997,531; U.S. Pat. No. 6,080,148; U.S. Pat. No. 6,224,566; and U.S. Pat. No. 6,231,568, which are fully incorporated herein by reference.

### BRIEF DESCRIPTION OF THE DRAWINGS

The many embodiments of devices suitable for the practice of the methods of the present invention are further illustrated by the accompanying drawings that form part of the specification, and in which like numerals are employed to designate like parts throughout the same. In the drawings:

FIG. 1 is a schematic plan view of one embodiment of an apparatus for practicing the methods of the present invention, showing a percutaneous catheter, the distal end portion of which is positioned against the endocardium of the heart, with an inner cannula/needle/optical fiber inserted into the myocardium. The proximal end portion of the outer catheter is removably attached to a housing and the inner catheter/cannula is attached to a mechanism which controls its insertion into and withdrawal from the myocardium. A separate mechanism advances the plunger of a syringe for delivering bone marrow or a bone marrow suspension, which may be enriched with stem cells collected from peripheral blood, during a portion of the inner cannula's insertion into the myocardial wall.

FIG. 2A is a schematic diagram showing further details of the apparatus and supporting features shown in FIG. 1, showing in partial cut-away the fluid communication through the lumen of the inner catheter.

FIG. 2B is a partial cutaway sectional view of one embodiment of an inner catheter/needle/optical fiber device for practicing the methods of the invention (the positioning of an optical fiber within the lumen of the movable inner cannula is shown with the inner catheter being cut-away). In this particular embodiment, a short length of syringe needle which can be crimped down and firmly attached to the optical fiber is shown. As shown in all FIGURES, the single optical fiber may also be a suitable bundle of smaller diameter optical fibers.

FIG. 2C is a cross-sectional view of a needle/optical fiber device of the invention, wherein the needle is crimped at two opposite points (for example 3 and 9 O'Clock) so as to firmly fix the optical fiber within the bore of the needle, allowing fluid communication via the remaining elliptical space of the bore (in the example shown, at 12 and 6 O'Clock).

FIG. 3 is a cross sectional view taken along line 3-3 shown in FIG. 2A, showing a cross section of one embodiment of the outer catheter with an inner movable catheter through which bone marrow, bone marrow suspension or other therapeutics can be delivered to the site of pocket formation, and the optical fiber positioned within this inner catheter, with one or more deflecting wires for articulating the distal end of the catheter. Also shown are optional anchoring wires and optional ultrasound signal wires.

FIG. 4 is a cutaway cross sectional view of the distal end portion of one percutaneous catheter suitable for practicing the methods of the invention, showing the tip of an outer catheter positioned against the endocardial surface of the heart. Movably disposed within the outer catheter is an optical fiber within the lumen of an inner cannula terminating in a syringe needle.

FIG. 5 is similar to that of FIG. 4, except the optical fiber/needle assembly has been advanced into the myocardium, and a pocket has been formed by laser energy from the laser (not shown), with bone marrow or a bone marrow suspension being injected into the pocket formed in the heart muscle as the needle/optical fiber assembly is being withdrawn

FIG. 6A is a cross sectional view of a heart wall, showing the pocket with the bone marrow or bone marrow suspension within the myocardium, created after injection from within the heart chamber during a percutaneous procedure.

FIG. 6B is a cross sectional view of a heart wall, showing the pocket of FIG. 6B being created from the exterior or epicardial surface of the heart.

FIG. 7A is a schematic plan view, similar to that of FIG. 1, of one embodiment of an apparatus for practicing the methods of the present invention, showing a handpiece and metal cannula for use in an open chest or endoscopic procedure, the distal end portion of which is positioned against the epicardium of the heart, with an inner catheter/fiber/needle assembly inserted into the myocardium. The proximal end portion of the outer catheter is removably attached to a housing and the inner catheter is removably attached to an actuator mechanism (stepper motor), which controls its insertion into and withdrawal from the myocardium. A separate actuator mechanism advances the plunger of a syringe for bone marrow or bone marrow suspension delivery during a portion of the fiber/needle assembly's insertion into the myocardial wall. For an endoscopic procedure, the length of the metal cannula extending distally from the handpiece can be greater than that depicted.

FIG. 7B illustrates the insertion of a device for practicing the methods of the present invention by open chest or endoscopic procedure through a puncture between the ribs, depicting the abutment of a flange against the epicardium and the injection of the bone marrow or bone marrow suspension into a pocket in the heart muscle formed by the laser energy as the fiber/needle assembly is being withdrawn from the myocardium.

FIG. 7C illustrates the fiber/needle assembly of the device inserted, without lasing, partially into the heart wall from the epicardium of the heart.

FIG. 7D illustrates the fiber/needle assembly of the device having been inserted, while lasing, through the remainder of the heart wall into the chamber.

FIG. 7E illustrates the channel created by the emission of laser energy from the device of FIG. 7D, with bone marrow or suspension thereof deposited in the channel.

FIG. 7F illustrates a wider channel into the endocardium produced by double lasing in that area.

FIG. 7G illustrates the fiber/needle assembly of the device having penetrated, while lasing, into the myocardium from the inside of the left ventricle.

FIG. 7H illustrates the channel created by the device of FIG. 7G, with bone marrow or a suspension thereof deposited in the channel.

FIG. 7I illustrates a wider channel produced by double lasing in the myocardium.

FIG. 7J illustrates the placement of alternating rows of channels and pockets created in the heart wall.

FIG. 8 is a diagram, showing a typical ECG wave form of a patient, upon which the window of time between heart beats for insertion of the inner cannula needle into the myocardium and injection of bone marrow or bone marrow suspension is defined by the operator locating Bar 1 and Bar 2. The mechanical insertion, advancement while lasing, lasing cessation and, during partial withdrawal, the injection of bone marrow or bone marrow suspension into the myocardial wall, and complete withdrawal from the heart wall is shown and are timed such that the procedures of the invention are synchronized to occur a selected time after the “r” wave of the patient's ECG, to fall within diastole.

FIG. 9A diagrams a further alternative embodiment of a needle/optical fiber tip assembly of the invention, for practicing the methods of the invention showing the inner catheter overlapping the proximal end of the needle, with the fiber fixed within the needle and maintaining fluid communication through the needle. The arrows indicate the approximate location of the cross-sectional views depicted in FIGS. 2B-2C, 3 and 11-13.

FIG. 9B diagrams an alternative embodiment of a device of the invention in which the proximal end of the needle is shaped with a flange to which the distal end of the inner catheter is attached, so as to minimize perturbation of the outer surface of the inner catheter/needle, with the optical fiber fixed within the lumen, which allows fluid communication. The arrows indicate the approximate location of the cross-sectional views depicted in FIGS. 2B-2C, 3 and 11-13.

FIG. 10 illustrates the proximal end of the inner catheter of the device, which is held in place by fluid-tight attachment to the optical fiber. The optical fiber is not large enough to completely fill the lumen, and thus the inner cannula retains at least one fluid communication channel. A fluid communication means between the lumen of the inner cannula and an external source of bone marrow or bone marrow suspension is shown having, in this embodiment, a Luer lock at the end of a rigid, or flexible post (“y” or “t” connector) in fluid-tight attachment to the inner cannula.

FIG. 11 is a cross sectional view showing an alternative embodiment of a needle/fiber optic device for practicing the invention, wherein one or more channels created in the jacket or buffer coating of the optical fiber permit fluid communication through the lumen of the inner catheter and needle.

FIG. 12 is a similar cross sectional view as shown in FIG. 11, depicting optical fiber covered by a needle, and the fluid communication channels being interspersed between flanges on the inner surface of the bore of the needle, which flanges fix the optical fiber in place.

FIG. 13 is similar to that depicted in FIG. 11, showing an alternative embodiment of the needle/metal tip-optical fiber of the invention, showing fluid communication channels within the bore of the needle, between flanges, which fix the optical fiber within the needle, and side ports to allow lateral exit of liquid.

FIG. 14 is a cutaway cross sectional view of an additional embodiment of a device suitable for the practice of the present invention showing that the proximal end of the inner cannula is in fluid connection with a source of therapeutic agent by means of a gasket, and the optical fiber within the inner catheter is in communication with a source of laser energy. Inner and outer metal tubes enable the inner catheter/needle/fiber assembly to be advanced and withdrawn by a stepper motor mechanism.

FIG. 15 is a schematic diagram showing an alternative embodiment of another device suitable for the practice of the methods of the invention, in which the bone marrow or bone marrow suspension is delivered by a pump into the inner catheter. This pump embodiment can also be adapted for injection into the heart wall during open surgery or in a endoscopic procedure through a puncture between the ribs, as well as in a percutaneous procedures.

FIG. 16 is a cross section view of the distal end portion of an alternative embodiment of a device of the invention in which ultrasound transducers are used to enable the operator or a micro-processor to determine the thickness of the myocardial wall. A movable obturator is inserted within the tube which, when advanced, injects bone marrow or a bone marrow suspension. No laser energy is used with the device.

FIG. 17 depicts a view similar to that of FIG. 16, showing a rod with an “o” ring to effect a seal with the inner catheter, which is used as a plunger to eject bone marrow or a bone marrow suspension from the needle, aliquots of which may be separated by gaseous bubbles of a predetermined size. No laser energy is utilized with this device.

FIG. 18 illustrates a conventional syringe manually or mechanically actuated to eject a desired amount of bone marrow or a suspension thereof from the needle. No laser energy is used with this device.

FIG. 19 illustrates another embodiment of the invention with a short length of syringe needle affixed to the distal end of the inner catheter by adhesive or the like. Again, no use of laser energy is entailed in this embodiment. One or more pellets of bone marrow extract, optionally in a pointed end or bullet shaped configuration, are lined up within the inner catheter. The inner catheter/needle assembly is advanced a selected distance into the tissue, organ or heart wall manually or by a first advancement mechanism. The obturator is advanced manually or by a second advancement mechanism a distance necessary to expel one or a desired number of the pellets.

FIG. 20 is a schematic drawing of an alternate embodiment of the present invention, which includes a mechanical means to advance the inner catheter/needle assembly through the outer catheter, whose distal end can be positioned on the endocardium of the heart, an organ or other tissue. A separate mechanical means advances the plunger of a syringe to inject bone marrow or a suspension thereof into the tissue. No optical fiber or laser source is used in this particular embodiment.

FIG. 21 is a schematic representation of an alternate embodiment of the present invention. As in FIG. 20, a mechanical means is used to advance the inner catheter/needle assembly through the outer catheter, which can be positioned on the exterior surface of the heart, an organ or other tissue. A separate mechanical means advances the plunger of a syringe to inject bone marrow or a suspension thereof into the tissue. No optical fiber or laser source is used in this embodiment. As in FIG. 20, movement of the inner catheter/needle assembly and the plunger of the syringe is controlled, when the activation button is pressed, by a microprocessor, which can optionally synchronize these actions with the patient's ECG.

FIG. 22 illustrates the basic components of a kit for extraction and preparation of bone marrow or a bone marrow suspension.

FIG. 23 shows a human stem cell lodged at the bifurcation of an artery. The arrow indicates the direction of flow.

FIG. 24 shows a human stem cell encapsulated in a liposome, which is lodged at the bifurcation of a larger artery than shown in FIG. 23.

FIG. 25A illustrates intercellular bone marrow material encapsulated in a liposome, which is lodged at the bifurcation of a smaller artery than is shown in FIG. 23.

FIG. 25B shows the liposome encapsulated intercellular material of FIG. 25A, with the surface of the liposome layer facing the direction of fluid flow being decomposed by the pressure being exerted thereon, and the intercellular material being released into the blocked artery.

FIG. 26 is a graphical presentation of data illustrating effective cardiac tissue repair using autologous adult bone marrow.

### DETAILED DESCRIPTION OF PREFERRED EMBODIMENTS

Bone Marrow

The constituents of bone marrow and their development are known and are described in Cecil, Essentials of Medicine, Fourth Edition, by Andreole, et al., pages 355-357 (1997). In the normal adult, blood cells arise in the bone marrow. Normal bone marrow consists mainly of stem cells, hematopoietic precursors of granulocytes, including myeloblasts, (which are highly replicative), as well as erythrocytes, lymphocytes, plasma cells, monocytes, precursors of platelets (megakaryocytes) and intercellular liquid, altogether making up about 60% of the marrow mass. The remaining 40% of bone marrow includes fat cells and supportive elements (blood vessels, red blood cells, and fibroblasts).

The cytoplasm of granulocytes and myelocytes is rich in ribonucleic acid (RNA). As the granulocytes and myelocytes mature, granules appear containing microbial components (defensins, permeability-increasing proteins, lysozyme, myeloperoxidose and hydrolytic enzymes (neutral and acid protease). Later specific cell granules appear (neutrophilic, cosinophilic or basophilic), collectively myelocytes, which mature into metamyelocytes or segmented neutrophils.

As erthrocytic precursors mature, the rubriblasts, (which are rich in RNA), become reticulocytes, and move to the spleen to mature for one day or so before entering the peripheral blood stream.

Megakaryocytes, precursors of platelets, begin as a blast cell and differentiate into platelet-like domains. Mature megakaryocytes contain an amount of DNA equal to that of 32 to 64 normal cells.

Lymphocytic cells differentiate under the influence of a variety of cytokines, particularly interleukin-7 (IL-7). These cells mature in the thymus, spleen and lymph nodes where, under specific controls, they further differentiate into the panoply of the immune system's lymphocytes.

Human mesenchymal stem cells (hMSCs) give rise to marrow stromal cells which produce the spongy matrix of the bone marrow. Marrow stromal cells produce a spectrum of growth factors and other molecules that regulate the proliferation, differentiation and maintenance of human stem cells and their precursors.

Stem cells form mixed “GEMM” (granulocytes, erythrocytes, megakoryocytes and monocytes) colonies under the influence of the granulocyte colony stimulating cytokine GM-CSF. Macrophages split from erythrocytes-megakaryocytes, and further are split under the influence of the granulocyte colony stimulating cytokine G-CSF, into granulocytes and monocytes. The split into erythrocytes and megakaryocytes is influenced by two cytokines, erythropoietin (EPO) and (presumably) thrombopoietin (TPO). Platelets develop (presumably) under the influence of IL-11 and TPO, although their action and the function of related unknown elements is not clear.

Liu, J. et al., in “Immunoelectron Microscopic Localization of Growth Factors and Other Markers in Human Long-bone Marrow Cultures”, Chin. Med. Sci. J. 11(3):129-35 (1996), showed that bFGF (FGF-2), GM-CSF and G-CSF were present in bone marrow, based on intense labeling for electron microscopy. Double labeling of heparin sulfate proteoglycans and CM-CSF showed binding of the growth factor, bFGF, to the extracellular matrix.

Bankes et al., in “Release of the Angiogenic Cytokine Vascular Endothelial Growth Factor (VEGF) from Platelets”, Br. J. Cancer 77(6):956-64 (1998), showed that VEGF was contained in platelets and was released upon their activation during coagulation. They also found VEGF within megakaryocytes and other cell types in bone marrow.

Möhle, R. et al., in “Constitutive Production and Thrombin-induced Release of Vascular Endothelial Growth Factor by Human Megakaryocytes and Platelets”, Proc. Nat. Acad. Sci. USA 21:94(2) 663-68 (1997), showed that co-culture of bone marrow microvascular endothelial cells with hematopoictic progenitor cells results in proliferation and differentiation of megakaryocytes, and these cells and CD41a⁺ cells secreted VEGF-121, 165 and 189, primarily the 165 isoform, and that thrombin stimulated the release of VEGF in 30 minutes.

Maloney et al., in “In Vitro Release of Vascular Endotheleal Growth Factor during Platelet Aggregation,” Am. J. Pysiol. 275(3P2):H1054-61 (1998), demonstrated that, during aggregation, platelets release VEGF.

Thus, bone marrow has been shown to contain growth factors and cells containing the genes that cause the expression of angiogenic growth factors, as well as a variety of presently unidentified angiogenic agents, promoters, enhancers, maturing agents, signalling agents and other substances, in addition to undifferentiated stem cells. Such stem cells, in the presence of the cytokines present in bone marrow, develop into blood cell lines. Injected into tissue, under the influence of local tissue specific cytokines, stem cells differentiate into the cells of the tissue into which they are injected, including epithelial cells, smooth muscle cells, neurons, etc., as a result of signalling agents that seek or potentiate the development of needed cell types.

The average adult body contains 10¹³ cells, all derived from one embryonic zygote. Embryonic stem cells may self renew or differentiate, based on extrinsic signals found in their environment. The decision to self renew (not differentiate) is dictated by leukemia inhibitory factor or LIF (Hilton & Gough, 1991; Smith et al, 1992). Section 112 of The Biomedical Engineering Handbook, titled The Biology of Stem Cells, Jordan, and Van Zant, describes how embryonic stem cells are totipotent, able to implement every possible program of gene expression, into all of the adult cell phenotypes.

Stem cells may double as many as 5000 times or more over a period of 70 years. The decision to differentiate is usually dictated by a growth factor or other cytokine, although this process is presently poorly understood. After differentiation, approximately 50 cell divisions occur before senescence, but a larger number of divisions may occur for certain cell types, such as intestinal epithelial stem cells, which may extend to several thousand divisions. This may be due to embryonic stem cells having the ability to regenerate their telomeres by expressing an enzyme (telomerase).

Some types of embryonic stem cells are committed to differentiation by platelet-derived growth factor (PDGF) and bFGF (Wren et al, 1992).

Tissue specific stem cells are pluripotent, but not totipotent. Tissue specific stem cells are found in the liver, nervous system, hematopoietic system and skeletal muscle cells, among others.

Stem cells in the adult may also be obtained by extraction of bone marrow or collected from peripheral blood by means known in the art. The numbers of stem cells can be stimulated by exposure to appropriate growth factors, including LIF. A small bone marrow specimen, taken under local anesthesia in an outpatient setting, or stem cells collected from peripheral blood by apheresis, as known in the art, can be expanded outside the body into a larger number of stem cells, particularly in the presence of transforming growth factor beta (TGFb), for reimplantation after one or more courses of chemotherapy. Stem cells may also be cultivated in vitro for subsequent administration to a patient.

Takahashi, et al., “Ischemia- and cytokine-induced mobilization of bone marrow-derived endothelial progenitor cells for neovascularization”, Nature Medicine, 5(4):434-438 (April 1999) report that GM-CSF stimulated production of endothelial progenitor cells and improved hind limb neovascularization in animals with hind limb ischemia. Stem cells and GM-CSF are found in bone marrow. In a press release issued Jul. 15, 1999 (Business Wire), Cyclotherapeutics, Inc. reported on work by Fricker, and Bjorklund, which showed that human embryonic neural stem/progenitor cells cultured with certain neurotropic factors, when implanted into rodent brains, migrated from the site of transplantation and integrated into host neuronal brain tissue in the hippocampus, olfactory bulb and other regions of the brain, by differentiating into neuronal cell types in response to cues in the tissues in the areas of the brain into which they migrated. Mature, differentiated brain cells do not have the ability to migrate.

In an article in the Orange County Register on Jul. 7, 1999, Stephen Bartelmez, M.D., reported on the ability to grow bone marrow-derived mouse stem cells for several months in the presence of thromboprotein (TPO), fibroblasts, megakaryocytes and endothelial cells, which may provide growth factors and other stimulatory, maturation and signalling agents. Stem cells in laboratory culture usually die after one month.

McKay, R. D. G. et al., reported in “Embryonic Stem Cell-Derived Glial Precursors: A Source of Myelinating Transplants”, Science, 285:754-756 (July 1999) that embryonic stem cells can repair nerves in the spinal cord and brain. However, the use of embryos is controversial and may be constrained by limitations on federal support of research and proposed legislation. If the use of embryonic stem cells is ultimately determined to be legal and ethical, they may be used to supplement the capacity of bone marrow to repair tissue. Ideally, cord blood of an individual could be frozen at birth and stored for future use by that person for such purpose, avoiding any rejection mechanism.

Creating a suspension of stem cells, isolated from the patient's bone marrow or collected from his/her peripheral blood and grown in a laboratory, as described above, can be injected in a growth enhancing amount into the heart, brain or a variety of other living tissues of the body, where the injected stem cells migrate to and differentiate into types of cells that are needed by the tissue into which they are injected (blood vessels if ischemia is present, myocytes if cardiomyopathy is present, neuronal tissue if electrical or synapse dislocations are present, islets and beta cells in the pancreas in diabetics, etc.) all under the influence of local cytokines, which may be present or are expressed as a response to local needs.

Likewise, a liquid suspension of bone marrow can be so injected to obtain a similar effect. A suspension of bone marrow in a physiologically compatible liquid, enriched by the addition of stem cells, collected and grown as described above, achieves an even more desirable effect when administered in a growth enhancing amount to a tissue to be repaired.

In this manner, a variety of organs and tissues can be revascularized, repaired or revitalized in a physiologically compatible manner, rather than by administering one or more costly agents made by recombinant technology, which may have unanticipated adverse effects. As described by Harawala, et al., in “VEGF Improves Myocardial Blood Flow But Produces EDRF-Mediated Hypotension in Porcine Hearts”, J. Surg. Res., 63:77-82 (1996), the administration of VEGF was shown to improve myocardial blood flow, but produced a significant depression in blood pressure, which in turn caused the death of several of the animals in the study.

A liquid suspension of autologous (host) bone marrow, autologous stem cells, isolated, collected and grown as described above or autologous bone marrow enriched by the addition of autologous stem cells, as well as autologous bone marrow, by itself, compressed into a pellet or enriched with autologous stem cells, are hereinafter collectively referred to as autologous growth agents (AGA), the preferred growth agents.

To revascularize cardiac tissue, i.e., heart tissue, an effective amount of AGA is injected, with the heart arrested, into tissue such as the heart muscle with a syringe or pellet injector, or into a space created in the heart wall by a laser, as described herein. Such a procedure is useful to treat (a) ischemic heart disease caused by one or more blockages in the coronary arteries by causing angiogenesis, (b) congestive heart failure (CHF) by the creation of cells secreting adenylyl cyclase to enhance cAMP signalling, or guanylyl cyclase to enhance gAMP signalling, both of which cause smooth muscle cell relation and dilation of veins and arteries, as well as the conversion of stem cells into myocytes and/or nerves, (c) cardiomyopathy by the transformation of stem cells into myocytes and nerve cells, and (d) heart wall motion abnormality by restoring the viability of a portion of the heart wall which does not function, or only partially functions, due to the presence of scar tissue from an earlier acute myocardial infarction, by injecting an AGA into the scar tissue and surrounding area, creating both angiogenesis and the transformation of stem cells to myocytes, nerves and other supporting cell types. Injected into a brain artery, the brain, or spinal column, in addition to angiogenesis, AGA causes the production of needed brain or neuronal cells, for example, to treat an ischemic stroke. Likewise, injected into other organs or tissues, whatever cell type is needed is created by naturally occurring “demand” cytokines in the tissue.

An AGA (therapeutic fluid) can be administered, for example, in an aqueous or non-aqueous physiologically compatible liquid suspension (vegetable oil, aliphatic acid glyceride, ester of an aliphatic acid, or propylene glycol), encapsulated in liposomes in a liquid suspension, in a solid or semi-solid form, with or without a vehicle which is solid at room temperature and melts (becomes liquid) at body temperature, in the form of a gel, ointment, or poultice, for application to the skin, scalp, a wound or surgical incision, as a suppository with a vehicle such as cocoa butter or polyethylene glycol (i.e., CARBOWAX®, available from Union Carbide Corporation), for bowel and lower intestinal applications or as a powder inhalant or aerosol for pulmonary applications.

Delivery of an AGA (therapeutic fluid) to the desired site can be accomplished, for example, by oral, parenteral, intravenous, intra-arterial, inter-pericardial, intra-pulmonary, intramuscular, interstitial, intrathecal, intracranial, intraperitoneal, transdermal, subdermal, intradermal, topical, and the like routes. It may be introduced into any tissue deficient in blood flow or normal cells, or into an artery suppling blood to such tissue.

An AGA can also include biologically compatible auxiliary substances, such as pH adjusting and buffering agents, tonicity adjusting agents, stabilizers, wetting agents and the like, by means well known in the art. An AGA can also be administered with agents that enhance nitric oxide (NO) levels (by enhancing NO synthase and release of NO), or agents that enhance prostacyclin levels (enhancing prostacyclin synthase and releasing prostacyclin) both of which enhance angiogenesis. NO enhancers include, for example, L-arginine, L-lysine, antioxidants such as tocopheral, ascorbic acid (vitamin C) or ubiquinone. NO synthase enhancers include tetrahydrobiopterin, sepiapterin and the like. Prostacyclin enhancers include, for example, eicosopentanoic acid, docosohexanoic acid and prostanoids, such as prostaglandin E₁ and its analogs, and the like.

When formulated in a gel or matrix, utilizing a sodium carboxymethylcellulose-based agent or the like, for example, an AGA can be injected or extruded by a syringe in a path from a point at or near a blood vessel above an occlusion to a point at or near the blood vessel below the occlusion, thus creating a pathway for formation of a new vessel around the blockage.

For wound healing or other topical applications, the AGA can be combined with a physiologically compatible carrier such as a hydrophilic colloid, or other physiologically compatible material that can maintain a moist environment.

The therapeutic dosage of an AGA depends upon the extent of the tissue to be revascularized or repaired in vivo, the nature of the tissue, and other factors, and varies widely from application to application.

Since pulsed laser energy (Holmium:YAG, Excimer, CO₂, etc.) has been shown to induce angiogenesis in the transmyocardial laser revascularization (TMLR) procedure, and since pulsed laser energy and the administration of an angiogenic gene or growth factor has been shown to be complimentary by producing an additive effect on an ischemic heart wall greater than TMLR or injection of the gene or growth factor alone, it is possible to achieve enhanced angiogenesis by first applying laser energy and then injecting the AGA into the ischemic tissue. The additive effect of the laser may be due to an increase in cell wall permeability produced by pulsed laser energy and its acoustic shock effect.

In addition, the use of laser energy at an energy level sufficient to vaporize tissue, prior to injection of the AGA, creates a pocket or space in the tissue surrounded by a thin zone of coagulated tissue, in which the AGA may reside. In the heart, for example, unless a pocket or space is created, an injected substance would be largely expelled during the heart's next contraction. The coagulated tissue will be phagotosized and/or absorbed by the body over a period of several weeks, giving the AGA time to exert its effect. Pulsed laser energy has also been shown to increase cell wall permeability, enabling growth factors or genes to more readily and quickly enter cells.

When applied to a beating heart, the injection of AGA can best be accomplished from either the epicardial surface (outside) or the endocardial surface (inside) of the heart with the TMLR system described in U.S. Pat. No. 5,913,853 U.S. Pat. No. 6,231,568 which are fully incorporated herein by reference.

To avoid expulsion of the AGA during the next few compressions of the heart, the AGA can be injected into a pocket or trap in the heart wall, as described in U.S. Pat. No. 6,224,566 which is fully incorporated herein by reference.

To avoid generating a life threatening arrhythnia, the creation of a TMLR channel or pocket, along with injection of an AGA should be performed during diastole, when the heart's electrical activitely is minimal, as described in U.S. Pat. No. 4,788,975 (Shturman), which is fully incorporated herein by reference.

Other forms of energy may be used to create a channel or pocket in the wall of the heart including radio frequency energy, a rotating burr, piezo-electric energy, focused ultrasound, microwave energy and the like, as more fully to described in U.S. Pat. No. 6,224,566, which is fully incorporated herein by reference.

Likewise, to treat peripheral artery disease as a result of atherosclerosis, an AGA may be injected into the leg muscles, as well as into the ankle, foot and other tissues, to create angiogenesis to stimulate the healing of ulcers and to treat peripheral atherosclerosis and gangrene.

To repair an area of the brain which has been damaged due to ischemic stroke, an AGA may likewise be injected intracranially, into an artery feeding the affected area, intrathecally or into the spinal fluid, to cause both angiogenesis and transformation of stem cells into needed brain, nerve and other supporting cell types.

To treat diabetes, an AGA may be injected into the pancreas or infused into an artery feeding the pancreas. End stage, renal disease and kidney failure may be treated by infusing an AGA into the renal artery or injected into the kidney.

To treat spinal cord damage or damage to any other organ or tissue, the injection of an AGA can induce angiogenesis and the formation of any needed cell types.

To stimulate the growth of hair in bald or balding men, an AGA may be injected subcutaneously in a pattern over the area to be treated, or it may be applied topically, with or without a skin-penetrant agent, as is known in the art, in order to create keratinocytes and other follicle cells, produce blood vessels to supply blood to the dormant follicle, and stimulate the growth of new hair. The administration of AGA may be complemented by the prior administration of pulsed laser energy to increase cell wall permeability and create additional angiogenesis.

The amount of bone marrow composition to be injected varies with the size of the organ or the mass of the tissue to be treated. For example, to revascularize the left ventricle of an adult human heart, about 1 cc to 10 cc or more of bone marrow may be extracted, filtered through one or more screens, declining from a pore size of about 0.4 mm to about 0.2 mm, partially liquified with a small amount of serum free cell culture medium (such as Stem Pro-34®, manufactured by Life Technologies, Inc., Grand Island, N.Y.), centrifuged, and the buffy coat removed by pipette. The extracted buffy coat may be suspended in phosphate buffered saline (such as that manufactured by Fisher Scientific, Inc.), utilizing about 3 to about 15 cc of phosphate buffered saline, preferably about 5 to about 12 cc, per cc of bone marrow extract. Approximately 0.01 cc to 0.5 cc, preferably about 0.03 cc to about 0.2 cc, of the suspension is injected at each site, with the injection sites spaced approximately 0.5 to 1.5 cm apart, preferably about 0.9 to 1.1 cm apart.

A similar amount of an AGA may be injected into leg muscles as described above to treat peripheral atherosclerosis, promote healing of ulcers and treat gangrene. Likewise, to treat baldness, a needle may be subcutaneously inserted, perpendicular or parallel to the surface of the skin, and one or more sites injected with an AGA as described above, or the suspension may be applied topically to the scalp, as well as to a wound or surgical incision.

If the area of baldness; the damaged area in the heart, brain or other organ; or the wound or incision area is smaller or larger; a proportionately smaller or larger amount of an AGA may be injected therein.

Growth Factors

The originally characterized form of VEGF (approximately 34-46 kDa) was about 20% identical with platelet derived growth factor (PDGF) A and B chains, including conserved CYS residues. Another close homolog called placenta growth factor (PlGF), on the basis of its original source, was also cloned and identified and shares 53% amino acid sequence identity with VEGF. It is thought that VEGF and PlGF may interact in similar fashion as PDGF A and B chains to form hetrodimer proteins. Bone marrow has a copious supply of platelets and PDGF and has been shown to contain VEGF.

Acidic Fibroblast Growth Factor (aFGF or FGF-1) and basic fibroblast growth factor (bFGF or FGF-2) was characterized and compared by Gimenez-Gallego et al., “Brain-derived Acidic Fibroblast Growth Factor: Complete Amino Acid Sequence and Homologies”, Science 230: 1385-1388 (1985), and has been found to induce angiogenesis. See Thompson et al., “Site-directed Neovessel Formation in Vivo”, Science 241:1349-1352 (1988); Folkman et al., “Angiogenic Factors”, Science 235:442-447 (1987). Bone marrow has been shown to contain aFGF and bFGF.

Hariawala et al., “Angiogenesis and the Heart: Therapeutic Implications”, J. R. Soc. Med. 90:307-311 (1997) discuss several possible applications of angiogenic factors in treating man. They note that gene therapy using viral vectors appears promising, but after taking up foreign DNA, the virally transformed abnormal cell is attacked and destroyed by the host's immune system, and its expression of the gene ceases. Autologous (host) stem cells and bone marrow are non-immunogenic.

Recently, injection of FGF-1 close to the vessels after the completion of bypass anastomosis was demonstrated to induce neoangiogenesis in the human heart. Schumacher et al., “Induction of Neoangiogenesis in Ischemic Myocardium by Human Growth Factors”, Circulation 97: 645-650 (1998). Bone marrow has been shown to contain aFGF of FGF-1.

Genes to Produce Therapeutic Enzymes

While more patients have survived an acute myocardial infarction (AMI or heart attack) due to available intervention and treatments, there has been an increase in the number of patients suffering from congestive heart failure (CHF), a weakening of the heart muscle, and cardiomyopathy. It has been recently reported that injection into the myocardium of genes allowing myocytes to produce the enzyme adenylyl cyclase (AC), apparently allowing the heart to beat stronger by stimulation of cAMP production, was beneficial in the treatment of CHF. See Gao, M. et al., “Increased Expression of Adenylylcyclase type VI Proportionately Increases Beta-adrenergic Receptor-stimulated Production of cAMP in Neonatal Rat Cardiac Myocytes”, PNAS(USA), 95(3):1038-43. Thus, the injection into the hearts of persons with CHE or cardiomyopathy, injection of an AGA, which has the ability to create cells with the genes to express AC, as well as to produce new blood vessels, myocytes and nerve cells, enables their own hearts to produce therapeutic levels of AC and grow myocytes, blood vessels and nerves, as needed. The production of AC by the heart would allow for immediate and localized stimulation of cAMP production that would be beneficial in stimulating stronger heart action in patients suffering from CHF and cardiomyopathy. Likewise, injection of any AGA has the ability to create cells with the genes to express guanylyl cyclase to enhance gBMP signalling and blood vessel relaxation, as well as to produce myocytes, etc.

The injection of an AGA into the heart wall in the area of an infarct resulting from an earlier AMI would, in addition to angiogenesis providing increased blood flow, repopulate the infarct area with myocytes and nerve cells created by differentiated stem cells, due to “demand” cytokines expressed by the heart tissue.

Bone marrow can be removed under local anesthesia on an outpatient basis from a person's hip or femur using commercially available devices, such as biopsy needles, aspiration needles or the like, such as described in U.S. Pat. Nos. 4,314,565, 4,366,822, 4,481,946, 4,486,188 and others.

To prepare a suspension of bone marrow, in addition to a variety of other procedures known in the art, the following steps are preferably taken promptly, after removal of a bone marrow aliquot from the body: (a) the bone marrow is passed successively through one or more filters or screens of appropriate mesh size, from about 0.4 mm down to about 0.2 mm; (b) the filtered bone marrow is partially liquified with a small amount of tissue culture media, preferably serum free; (c) the filtered and liquified bone marrow is centrifuged at a relatively low speed (about 200 Gs for approximately 10 minutes); (d) any fat present is removed by pipette or other suction means and the buffy coat is recovered (discarding the remaining red blood cells); and (e) the recovered buffy coat is suspended in 3 to 10 or more parts of phosphate buffered saline for each part of buffy coat. The produced suspension is stored at about 25° C. if it is to be used within about one hour.

As provided above, the isolated bone marrow suspension, i.e., recovered buffy coat, can be suspended in phosphate buffered saline. In one embodiment, this suspension is constituted by at least about one part by weight phosphate buffered saline to one part by weight bone marrow (either bone marrow alone or in combination with stem cells). Preferably, the isolated bone marrow composition is constituted by about three parts by weight phosphate buffered saline to one part by weight bone marrow (alone or in combination with stem cells).

If the bone marrow is not to be immediately injected, it is refrigerated and maintained at about 4° C. If it is to be used more than twelve hours after the suspension is made, a physiologically compatible hydrocolloid can be optionally added to stabilize the suspension.

Pellets of bone marrow may be made by following steps (a), (b), and (c) as described above, promptly after which pellets, each having a volume of about 0.01 cc to about 0.5 cc, preferably each having a volume of about 0.05 cc to about 0.2 cc may be formed of the AGA itself or by adding a biologically compatible vehicle, which optionally may be solid at room temperature and liquid at body temperature, and stored at 25° C. if to be used within about one hour, or at 4° C. if to be used after about one hour.

A small amount of stem cells may be collected from the patient's peripheral blood stream by electrostatic charge, antibody adherence or other means as known in the art. After separation from other blood components, the stem cells may be diluted and injected as described herein or used to enrich the bone marrow, bone marrow suspension or pellets.

If the collection of stem cells from the patient's peripheral blood can be affected several weeks or longer before the injection procedure, the population of stem cells in the patient's blood can be increased by administering transforming growth factor beta (TGFb), as described in U.S. Pat. No. 5,426,098, or by growing the stem cells in a tissue culture medium by means known in the art, with their growth being stimulated by the addition of TPD and/or growth factors, particularly transforming growth factor beta (TGFb) and others, such as FGF and VEGF if angiogenesis is the intended effect, nerve growth factor (NGF) if nerve repair is the intended effect, etc. In addition to separating stem cells in the peripheral blood stream by attracting them to appropriate antibodies, such as various anti-CDs (CD₂, CD₅₆, CD₅₈, etc.) and other means known in the art, such as charge-flow separation apparatus as described in U.S. Pat. No. 5,906,724, and by other means.

Stem cells, if injected into the bloodstream, often exit from the bloodstream into the marrow, where certain adhesion molecules that attract them are present and the microenvironment is favorable. However, if appropriate adhesion molecules are not present, to which stem cells must adhere in order to initiate a signal transduction pathway, the stem cells often die. Thus, even though stem cells are often about 10 microns or larger in diameter and lodge in small arterioles and capillaries, if the needed adhesion molecules are not present, they may not survive.

A number of adhesion molecules have been identified, including various vascular cell adhesion molecules (VCAMs), endothelial leukocyte adhesion molecules (ELAMs), molecules involved in leukocyte adhesion (MILAs) and others, such as those described in U.S. Pat. Nos. 5,186,931, 5,272,263, 5,367,056 and others. Since some of these can be cloned and produced using recombinant genetic engineering, the addition of appropriate adhesion molecules to extracted stem cells, bone marrow or a bone marrow suspension to enhance the stem cells attaching to the tissue in the injected area and migrating therein.

An AGA suspension may simply be infused into a coronary artery, with the stem cells lodging in small arteries and surviving therein. Particles of intercellular bone marrow material, containing growth factors, promoters, enhancers, etc., may likewise be infused into a coronary artery after being encapsulated in liposomes by means known in the art, such as described in U.S. Pat. Nos. 4,089,801, 4,229,360, 5,017,359 and others.

However, the liposomes may uniquely be intentionally oversized, at least about 7 microns or more in diameter, versus conventional 0.5 to 2 or 3 micron diameter liposomes, which are intended to pass through arterioles and capillaries. By oversizing the liposomes to about 7 microns or more in diameter, they will not pass through very small arterioles and capillaries, and their contents, when the liposome disintegrates, will be discharged in the small arterioles of the tissue, rather than passing through the capillaries into the general circulation.

Such disintegration will first occur at the surface of the liposome on which the blood pressure is being exerted. To avoid microinfarcts (blockages) in these vessels, some or all of the liposomes may be designed to melt or disintegrate in about 2-3 minutes or less.

Infusion of an AGA, intercellular material or stem cells into an artery supplying another organ (kidney, liver, pancreas, brain, etc.) or a tissue (skin, muscle, etc.) can result in the same benefit. Optionally, encasing them in liposomes will enhance the beneficial effect.

Human hematopoietic stem cells, which range from about 8.2 to 8.7 microns in diameter +/−1.2 microns, when injected into the heart wall or infused into a coronary artery, lodge in the arteries as they reduce in size or at a bifurcation. Other types of stem cells are even larger, and will lodge in larger vessels. If “demand” cytokines are present, the stem cells will migrate to the area of need and differentiate into the needed cell types. If no local cytokines are present, the stem cells die in about one month. Incorporating adhesion molecules, as described herein, can prompt such migration and differentiation.

Encapsulating stem cells in liposomes, by means known in the art, increase the diameter of the globule to about 10-15 microns or larger. If they are infused into a coronary artery or injected into the heart muscle, they will lodge in larger vessels, or at a bifurcation, higher up in the vasculature, where they can be more effectively employed. U.S. Pat. Nos. 4,089,801, 4,229,360, 4,235,871 and 5,017,359 describe methods and formulations for liposome encapsulation.

To enhance the adhesion of the liposomes to the tissue, arteries and capillaries in the injected area, the liposomes may be given a cationic positive (+) charge, as described in U.S. Pat. No. 5,676,954 and others. Commercial cationic liposome materials include Lipofectin® cationic liposome reagents, manufactured by Life Technologies, Inc., Grand Island, N.Y.

A bone marrow suspension may include a thixotropic material, such as microcrystalline cellulose and/or carboxymethyl cellulose sodium (such as manufactured by FMC Corporation under the trademark Avicel®), which causes a decrease in viscosity of the suspension while pressure is exerted during the injection process, thereby facilitating administration of a relatively viscous substance which returns to its original viscous state once deposited at the desired tissue site. In addition to the above, hydrocolloids such as polyvinylpyrrolidone are also suitable, in one preferred embodiment having a weight average molecular weight of no more than about 10,000 Daltons.

In one preferred embodiment, a pocket or channel suitable for receiving an AGA is formed by laser energy. The device used for this purpose encompasses a single optical fiber or a bundle of optical fibers, located within the lumen of a first catheter, such that fluid communication is possible through the space between the fiber(s) and the inner lumen of the catheter. The distal end of this first catheter, maybe modified so as to be made suitable for mechanically puncturing tissue, such as heart muscle or other tissue, by attaching thereto a sharp plastic or metal tip or short length of syringe needle. This attachment may be made by means of a flanged coupling and suitable adhesive, mechanical means (crimping) or both. When fixably attaching the catheter to the fiber/needle assembly at this distal end, the attachment is made such that fluid communication is maintained through the lumen of the first catheter and out of the lumen at or near the distal end of the metal or plastic tip or needle. The exit may be through the bore of the needle, out exit openings in the end of the metal or plastic tip, or via side ports in the distal end of the needle or tip. This first catheter, containing one or more optical fibers within the lumen, may also be suitably used in conjunction with an appropriate outer catheter for manipulation and use in an open chest or minimally invasive endoscopic procedure through a puncture between the ribs, with a thoracoscope for visualization, as well as in a percutaneous procedure, for example, inserted into the femoral artery in the groin through a guiding catheter into the left ventricle or other heart chamber.

Thus in a particular aspect of the present invention, the device encompasses a distal end needle/tip-optical fiber construct which provides for fluid communication through openings in the needle tip to the lumen of a first catheter, while allowing for a fixed attachment of the distal end of an optical fiber, or bundle of fibers within said tip.

Implementation of the procedures of the present invention by the operation of certain exemplary device embodiments suitable for practicing the method of the invention is further described below.

As described in U.S. Pat. No. 6,231,568, and incorporated herein by reference, the distal end of an optical fiber, whose distal end is encased in a short length of syringe needle and whose proximal end is optically coupled to a source of laser energy, is contained within an inner catheter in fluid communication with the space between the optical fiber and the attached needle. The fiber/needle/inner catheter may also be movably disposed within a flexible outer catheter, terminating in a handpiece, from which a metal cannula extends. The distal end of the metal cannula is placed against the outer surface of the heart, organ, or tissue for example, the epicardium of the heart in an open chest or endoscopic procedure.

The distal end of the metal cannula can optionally provide for a flange to enable the device to be pressed against the heart to counter the recoil force of insertion of the fiber/needle into the heart wall. When applied against the outer surface of the heart (epicardium), the fiber/needle is mechanically advanced through the metal cannula a first predetermined distance into the heart wall without emitting laser energy. The fiber/needle advances a second, predetermined distance into but not entirely through, the heart muscle while emitting laser energy, creating a pocket within the tissue or heart muscle. As the needle is being withdrawn the second predetermined distance, an AGA is injected into the pocket, after which the needle withdraws the first selected distance out of the tissue. The AGA is trapped in and remains substantially within the pocket, immediately after administration.

In a percutaneous procedure, in which the flexible outer catheter is advanced through the vasculature and the aortic valve through a guiding catheter, as known in the art, the distal end of the outer catheter is positioned against the inner or endocardial surface of a heart. The fiber/needle is then mechanically advanced a first predetermined distance from the outer catheter into the heart wall without emitting laser energy. The fiber/needle advances a second predetermined distance into, but not entirely through the heart wall while emitting laser energy, creating a pocket in the myocardium. Injection of an AGA occurs as the fiber/needle withdraws the second selected distance, and the fiber/needle then withdraws the first selected distance from the heart wall. The AGA substantially remains trapped within the pocket in the heart wall.

In a further preferred embodiment, the method of the invention is practiced on a beating heart with synchronization of the movement of the fiber/needle/inner catheter being timed to begin at a selected time after the “r” wave of the patient's electrocardiogram (ECG) and to conclude within diastole, when the heart's electrical activity is minimal and the risk of an arrhythmia is least. In both of the aforementioned procedures, the needle/tip insertion distances and operation of the device can be armed by the surgeon by pressing a button, or armed by abutment of the needle/fiber tip to the surface of the heart. Activation of the device occurs a selected time after the “r” wave of the patient's ECG is sensed by a microprocessor controller, which causes a stepper motor mechanism to partially advance the fiber/needle mechanically a first selected distance, without emitting laser energy, enables laser energy to be transmitted through the optical fiber while advancing the fiber/needle a second selected distance, ceases the transmission of laser energy through the optical fiber, injects a selected amount of an AGA as the fiber/needle is being partially withdrawn, ceases the injection of an AGA and completes the withdrawal of the fiber/needle from the heart wall.

If the heart is arrested, the device can be activated by pressing a button or by abutment of the device against the heart, depressing a lever or actuator.

The various features and embodiments of the claimed invention are further illustrated by the description of the preferred embodiments below.

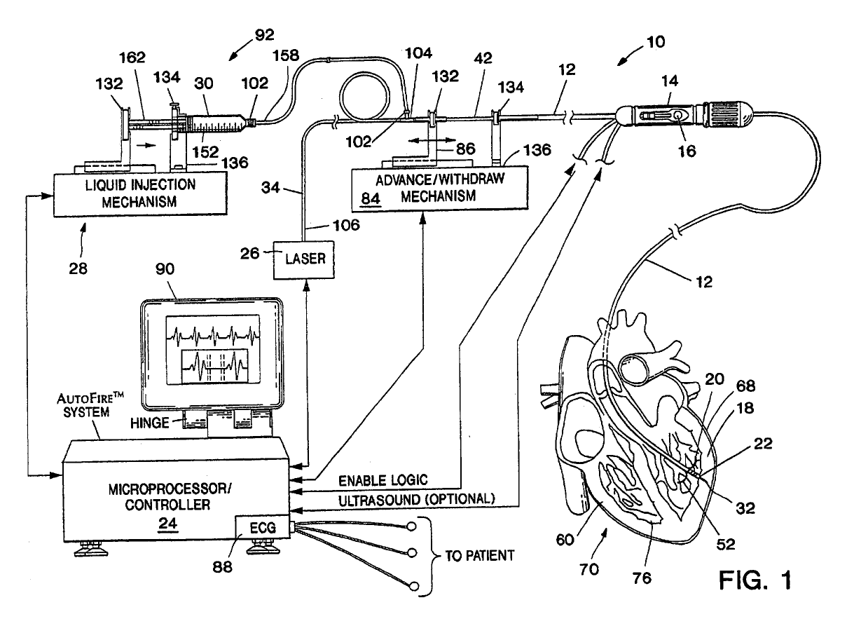

One example of a device 10 suitable for practice of this embodiment of the invention is illustrated in FIG. 1. The device 10 includes a flexible outer catheter 12 for insertion into the left ventricle, a handpiece 14, containing an activation button 16, placed about 20 to 90 cm from its distal end 18 of the catheter 12. A mechanism and wires 20 to articulate the distal end 18 of the flexible outer catheter 12 are attached to the outer catheter 12 about 40 to 100 cm from the distal end 18. Outer catheter 12 and encloses a moveable optical fiber/needle assembly encased in an inner catheter 42 (generally referred to as inner catheter/optical fiber/needle assembly 22).

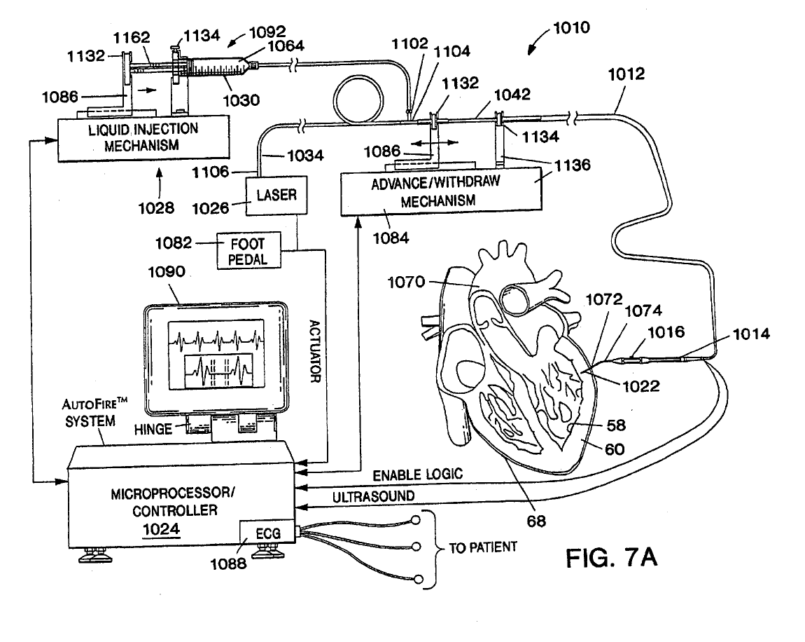

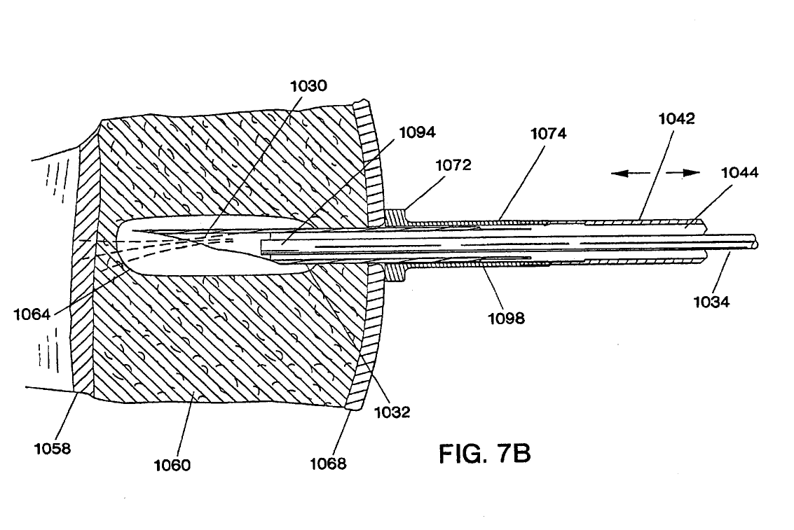

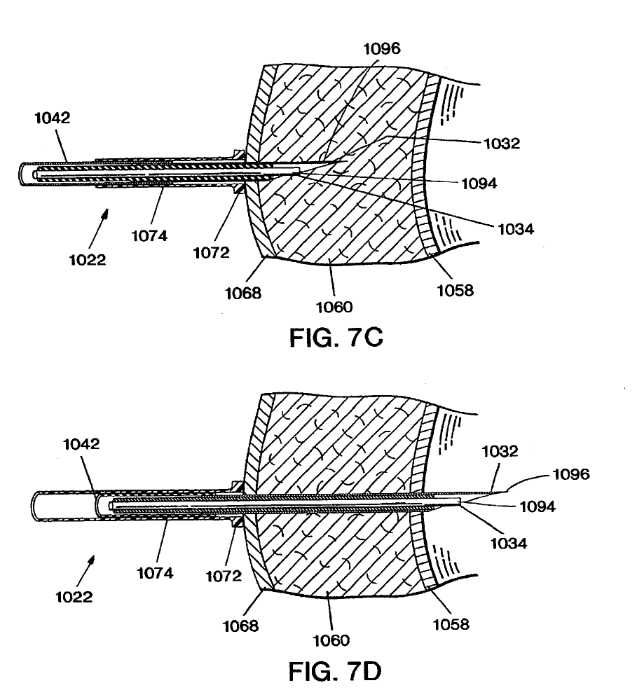

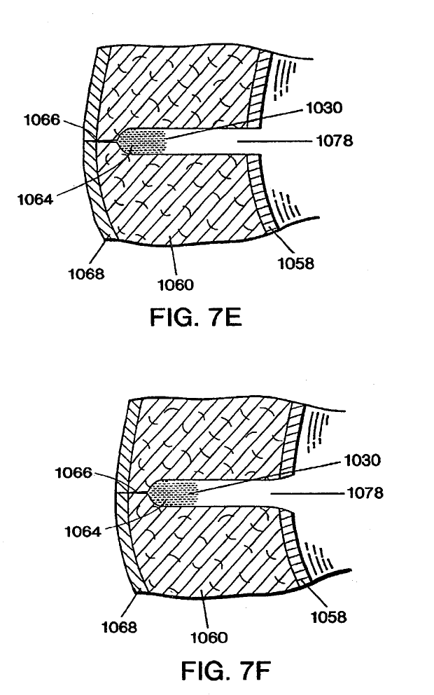

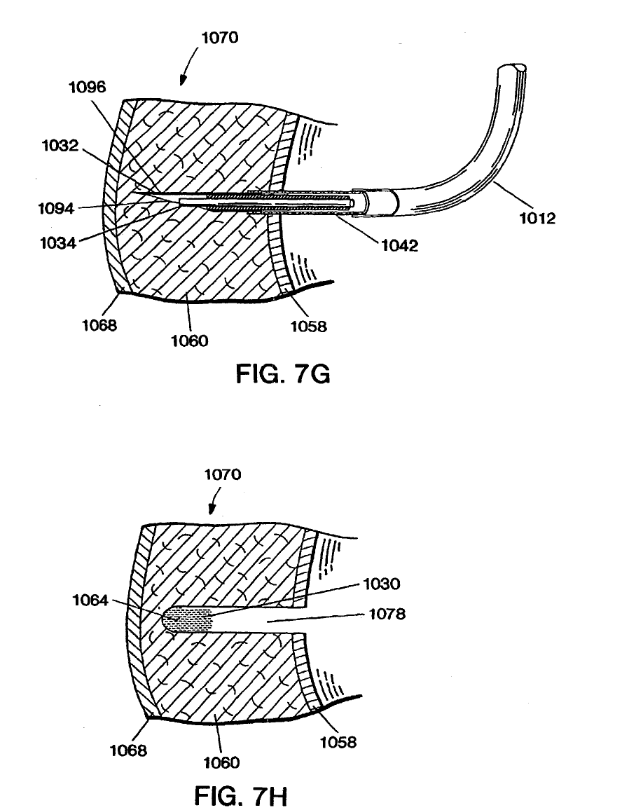

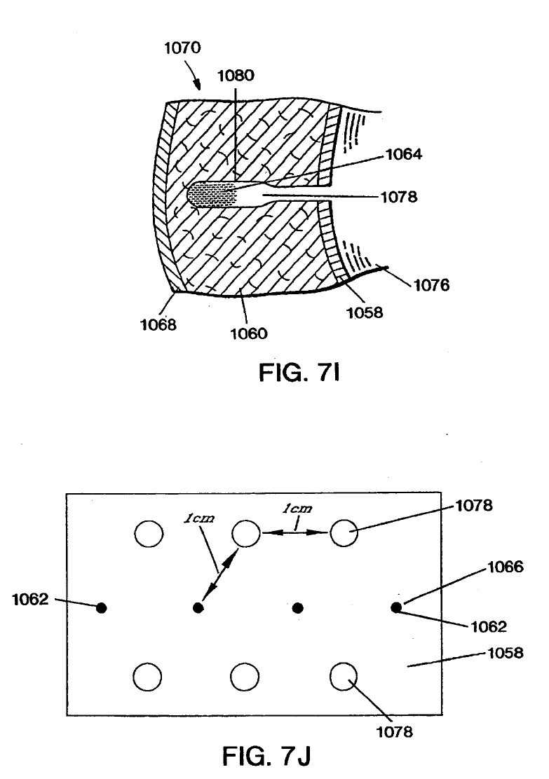

As seen in FIG. 1 (and FIG. 7A) a microprocessor controller 24 monitors the patient's ECG which is displayed on a monitor 90, and, at the times in the cardiac cycle selected by the operator, signals an advancement/withdrawal mechanism 84 to move the inner catheter/optical fiber/needle assembly 22, fires the laser 26, ceases firing the laser 26, signals the liquid injector mechanism 28 to inject liquid 30 as the inner catheter/optical fiber/needle assembly 22 is being withdrawn and completes the withdrawal of the inner catheter/optical fiber/needle assembly 22.

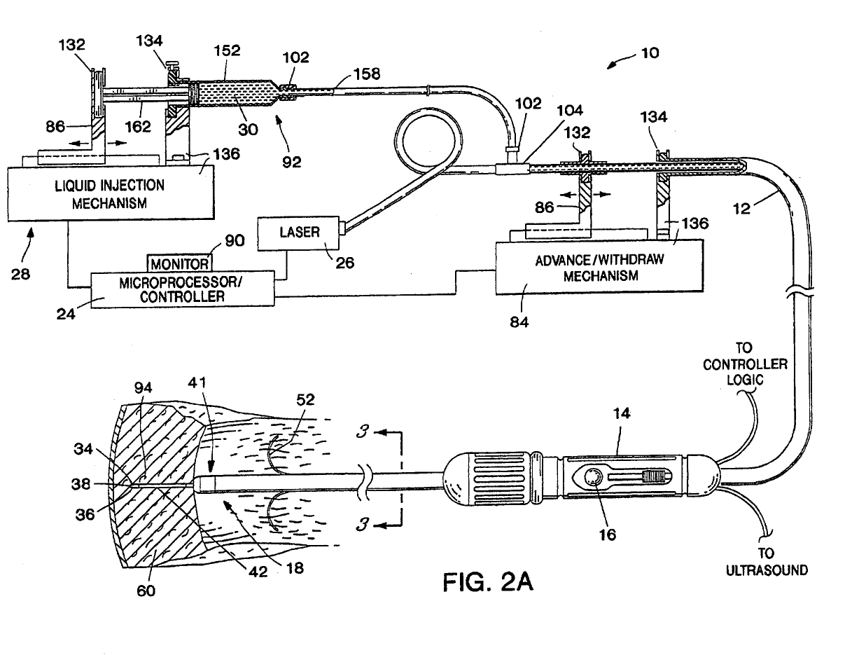

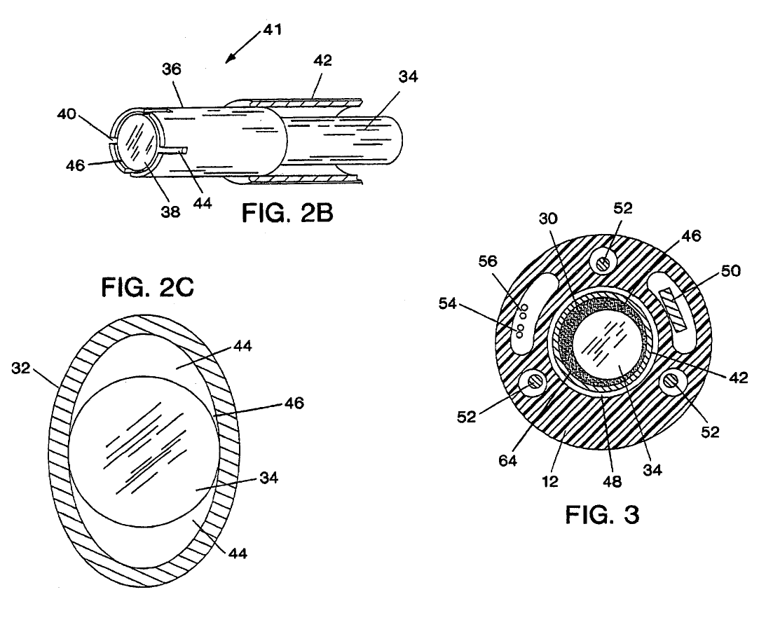

Optionally, as shown in FIG. 2A, instead of a short length of syringe needle 32, a metal tip or cap 36, which contains a lens 38 to expand the beam and one or more fluid ports 40, could be attached by crimping to the distal end of the optical fiber 34 together compressing the inner catheter/fiber/tip/lens assembly 41 in fluid communication with a syringe 152.

As seen in FIG. 2B, the lens 38 is contained in a metal tip 36, crimped to the optical fiber 34, with a fluid communication channel 44 between the lumen 46 of the metal tip 36 and optical fiber 34, with one or more exit parts 40 defined in metal tip 36. The inner catheter/optical fiber/metal tip/lens assembly 41 of FIG. 2B is described in U.S. Pat. No. 4,773,413 to Hussein et al., and incorporated herein by reference. The lens 38 diverges the beam to make a channel or pocket of a larger diameter than that of the optical fiber 34.

As seen in FIG. 2C, the needle 32 is crimped to the optical fiber 34 at, in one preferred embodiment, 3 and 9 O'Clock, and the resulting elliptical shape provides for fluid channels 44 at 6 and 12 O'Clock.

As seen in FIG. 3, the outer catheter 12 may contain several channels, in addition to a central channel 48 through which the inner catheter/optical fiber/needle assembly 22 may be advanced and withdrawn. At least one channel contains a deflecting wire 50 for manipulating the distal end 18 of the outer catheter 12. Additional channels in outer catheter 12 may optionally contain a second deflecting wire (not shown) to enable the device 10 to be articulate in the opposite direction, fixation or anchoring wires 52, wires 54 operably associated with an ultrasound transducer 57, enabling the operator to determine the thickness of the heart wall at the point of the distal end 18 of the outer catheter's 12 contact with the heart 70, and wires 50 to a strain gauge or other contact indicating means (not shown).

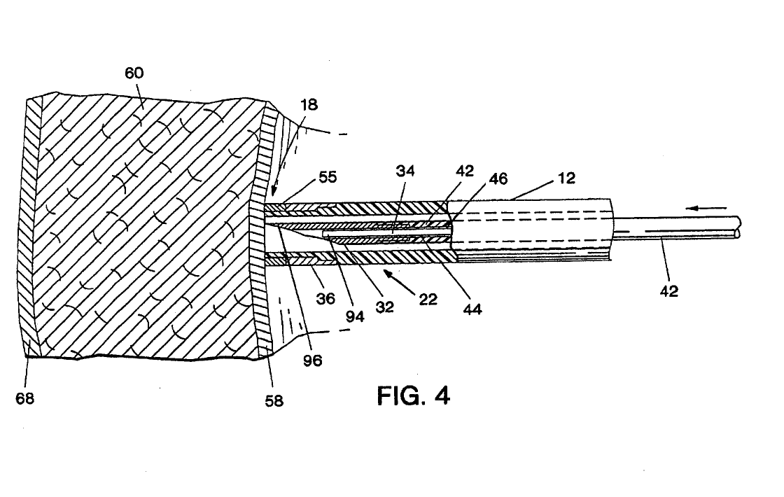

In FIG. 4, the distal, blunt end 18 of the outer catheter 12 is positioned against the inner surface of the heart wall (endocardium) 58 or other tissue.

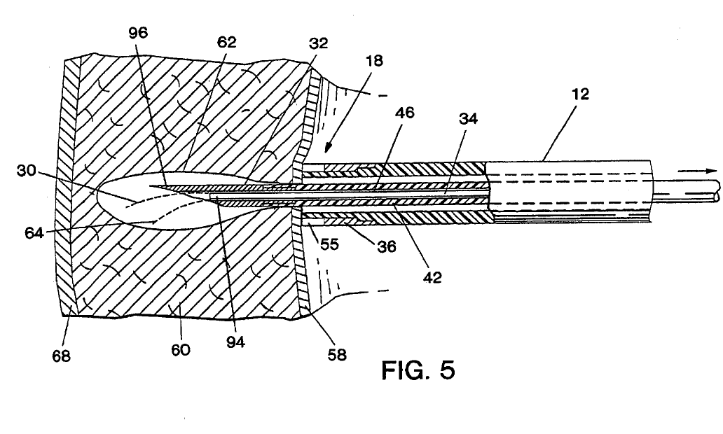

In FIG. 5, after mechanical insertion of the inner catheter/fiber/needle assembly 22 into the endocardium 58 without lasing, and advancement into the myocardium 60 while emitting laser energy creating a pocket 62, a fluid 30, in one preferred embodiment a therapeutic fluid 64, is injected as the inner catheter/fiber/needle assembly 22 is being withdrawn from the pocket 62. The inner catheter/fiber/needle assembly 22 is then withdrawn from the endocardium 58.

The preferred method of the present invention, in an open chest, endoscopic or percutaneous procedure, with the physician having determined the thickness of the heart wall at various levels by ultrasound prior to the procedure as is known in the art, calls for the partial insertion of the inner catheter/fiber/needle assembly 22 25% to 40% of the thickness of the heart wall by mechanical energy. The inner catheter/fiber/needle assembly 22 may be small in diameter, 14 gauge to 22 gauge, preferably 16 to 20 gauge, thus creating a small puncture wound which will easily clot or otherwise close. As the inner catheter/fiber/needle assembly 22 is advanced another 25% to 40% of the heart wall thickness, laser energy is emitted to create a pocket 62 within the heart wall. As the fiber/needle assembly 22 withdraws the second 24% to 40% of the heart wall thickness, injection of a therapeutic liquid (AGA) 64, into the pocket 62 is effected. While the injection of fluid 30 preferably occurs during the withdrawal of the inner catheter/fiber/needle assembly 22, it can occur with the inner catheter/fiber/needle assembly 22 while it is momentarily stationary within the pocket 62. Injection of fluid 30 is preferably via the space between the needle 32 and the optical fiber 34 by way of the inner catheter 42, which is in fluid communication with the tip of the needle 32. The fluid 30 may enter the inner channel via a tube from an external fluid source. The device 10 then withdraws 25% to 40% of the first distance out of the heart wall.

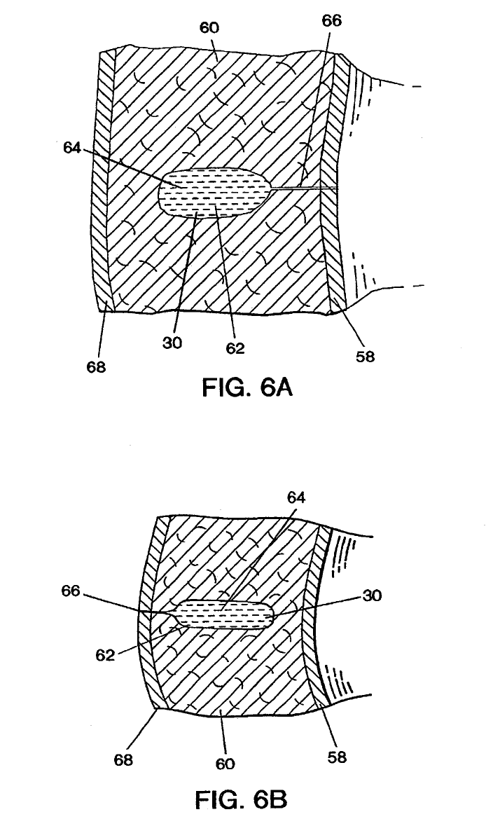

FIG. 6A is a drawing illustrating the resultant pocket 62 and needle puncture 66 formed in the myocardium 60 containing the therapeutic liquid (AGA) 62, after percutaneous treatment from the inside or endocardium 68 of the heart 70 wall. The tissue effect of performing the procedure from the outside or epicardium 58 of the heart, through an opening in the chest of a puncture between the ribs (not shown), is similar to that done percutaneously, however the wound is oriented in the opposite direction, originating from outside the heart 70. Because of the needle 32 leaves only a needle puncture 66 in the epicardium 68, which quickly clots or seals, bleeding is minimal. FIG. 6B illustrates the resultant pocket 62 formed in the myocardium 60 containing the fluid (AGA) 64, when formed from the epicardium 68 in an open chest or endoscopic procedure.

As seen in FIG. 7A, an embodiment of an apparatus is illustrated for performing the treatment from the epicardium 1068, during an open chest operation or through a puncture between the ribs, with an endoscope (thoracoscope) through a second puncture for visualization (not shown), to produce the pocket 1062 containing the fluid (AGA) 1064 seen in FIG. 6B. Where appropriate, the last two digits in the 1000 series of numerals depicted in FIGS. 7A-7F are connected to elements which have the same function and/or structure as those described with regard to FIGS. 1-6.

The distal end 1018 of the cannula 1074 attached to an handpiece 1014 is pressed against the epicardium 1068 of the heart 1070 (for example the left ventricle) or other tissue by the surgeon. By manually activating the activation button 1016, the inner catheter/fiber/needle assembly 1022 is first mechanically inserted 25% to 40% of the heart wall thickness into the heart muscle. Laser energy is emitted as the inner catheter/optical fiber/needle 1022 advances a second 25% to 40% of the thickness of the heart wall. The emission of laser energy ceases and, as the needle 1032 is withdrawn the second 25% to 40% of the thickness of the heart wall, a therapeutic fluid (AGA) 1064 is injected into the pocket 1062 created by the laser energy in the heart wall, as shown in FIG. 7B. The inner catheter/fiber/needle assembly 1022 is then withdrawn the first 25% to 40% from the heart wall.

As shown in FIG. 7B, a metal cannula 1074 is pressed against the epicardium 1068. Cannula flange 1072 prevents the device 1010 from prematurely puncturing the heart wall in similar fashion to the operation of the inner catheter/fiber/needle assembly 1022 of a percutaneous device, the flexible, directable outer catheter 1012, containing the moveable inner catheter/fiber/needle assembly 1022, is positioned on the endocardium 1058 of the heart 1070, the inner catheter/fiber/needle assembly 1022 is inserted a first 25% to 40% of the thickness of the heart wall into the endocardium 1058, without lasing. Lasing commences as the inner catheter/fiber/needle assembly 1022 advances a second 25% to 40% of the heart wall. Lasing ceases and a liquid (AGA) 1064 is injected as the fiber/needle assembly 1022 withdraws the second 25% to 40% of the heart wall into the laser created pocket 1062 within the heart wall. The fiber/needle 1022 is then withdrawn in the first 25% to 40% of the heart wall, out of the endocardium 1058.

In both cases, once the inner catheter/fiber/needle assembly 1022 assembly is completely withdrawn from the heart 1070, the needle puncture 1066 will either clot or seal to retain the injected fluid (AGA) 1064 inside the pocket 1062 formed in the heart muscle wall.

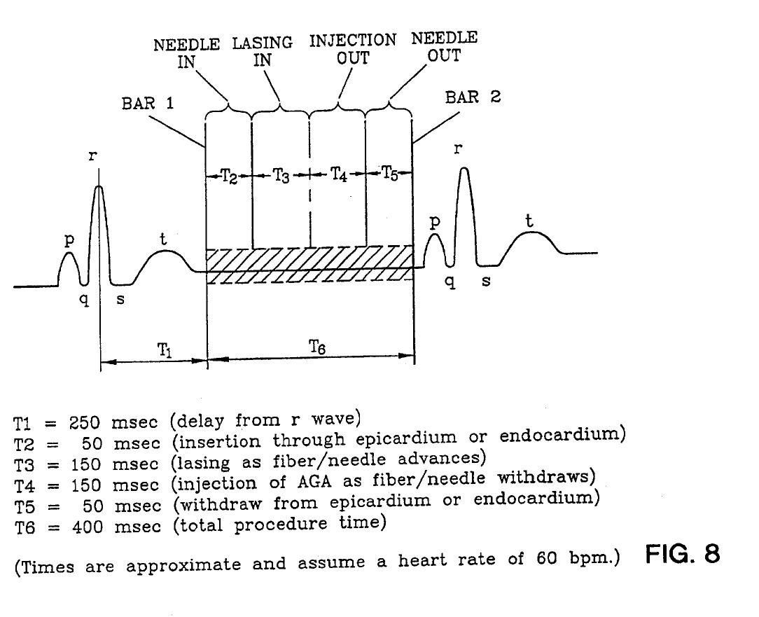

The procedures of the invention can be accomplished on a beating heart 1070, synchronized with the heart beat preferably occurring a predetermined period of time after the “r” wave of the patient's ECG, during diastole. Having earlier estimated the heart wall thickness in the area to be treated by ultrasound and having examined the patient's ECG pattern, the operator inputs the proper delay time from the “r” wave of the patient's ECG into a microprocessor (in one preferred embodiment microprocessor/controller 1024) with the distances and durations of each action of the advancement/withdrawal mechanism 1084, laser energy control system and liquid injection mechanism 28. Activation of the device 1010 can be manual, or automatically triggered by pressing the device 1010 against the heart wall, depressing an actuator, to effect insertion, lasing, cease lasing, injection and withdrawal, all within the course of diastole, through the surface of the heart 1070. As with the percutaneous apparatus and treatment, the preferred timing of treatment is shown in FIG. 8.

If used in an endoscopic procedure, a flange 1072 on a metal cannula 1074 should be approximately 4 to 8 mm in diameter, preferably about 5 to 7 mm, enabling it to be inserted through a small bore trocar puncture between the patient's ribs.

The apparatus for performing the procedure on an arrested heart during coronary bypass surgery or other open chest procedure is similar to that described above, except activation of the device is by pressing a button 1016 on a handpiece 1014, or by an actuator lever being depressed when the distal end of the metal cannula is pressed against the heart wall.

For an epicardial device, it is preferred that the needle 1032 or tip 1036 (either metal or plastic) be a 14-gauge to 22-gauge size, preferably 16 to 20 gauge, with a single 300 to 1000 micron diameter optical fiber 1034, or a bundle of 50 to 100 micron core diameter optical fibers therewithin. Typically, a 14 to 16 gauge needle 1032 or tip 1036 will have a 1000 micron core diameter or smaller optical fiber 1034 therewithin, and a 16 or 18-gauge needle 1032 or tip 1034 will have a 500 to 600 micron core diameter or smaller optical fiber 1034 there within. For an endocardial device, the needle/tip can be 16 to 22 gauge, preferably 18 to 20 gauge, with 500 to 600 or 365 micron core diameter optical fiber 1034, or a bundle of 25 to 100 micron core diameter optical fibers therewithin.

In FIG. 7C, in an open chest or endoscopic procedure, the flange 1072 of the metal cannula 1074 is pressed against the epicardial surface 1068 of the heart 1070 and the inner catheter/fiber/needle assembly 1022 is mechanically advanced a first distance, 25% to 40% of the way into the heart wall, without lasing.

As seen in FIG. 7D, the inner catheter/fiber/needle assembly 1022 is then advanced, while laser energy is being emitted, a second distance equal to the remaining thickness of the heart wall plus a third selected distance, about 1 mm to 6 mm, preferably 3 to 4 mm, into the chamber 1076 to assure a complete channel 1078 has been made (in case the physician's estimate of the heart wall is in error by a mm or two). Lasing may then cease, or it may optionally continue until the inner catheter/fiber/needle assembly 1022 has been withdrawn the third selected distance and not more than one-half the second selected distance, at which point the transmission of laser energy into the optical fiber 1034 ceases and the injection of a liquid (AGA) 1064 occurs as the inner catheter/fiber/needle assembly 1022 is being withdrawn the remaining portion of the second selected distance. The inner catheter/fiber/needle assembly 1022 is then withdrawn the first selected distance from the heart wall into the outer catheter 1012.

FIG. 7E illustrates the resultant channel 1078 in the endocardium 1058 into the heart chamber 1076, enabling blood from the chamber 1076 to perfuse the heart muscle. In FIG. 7E, lasing ceased after the inner catheter/fiber/needle assembly 1022 advanced the third selected distance into the heart wall.

As shown in FIG. 7F, in which lasing continued as the inner catheter/fiber/needle assembly 1022 was withdrawn the third selected distance and approximately 50% of the second selected distance, at which point laser transmission ceased. The wider channel 1078 in the endocardium 1058, resulting from the double lasing in that area, is less likely to close, enabling blood from the chamber 1076 to perfuse the heart wall for a longer period of time.

Also, increased cell wall permeability resulting from the acoustic shock wave produced by pulsed laser energy, enables the active constituents of the liquid (AGA) 1064 to penetrate the tissue more readily.

Likewise, a channel 1078 from the heart chamber 1076 into the myocardium 1060 and a liquid (AGA) 1064 deposited therein may be created in a percutaneous procedure. As shown in FIG. 7G, the distal end 1018 of the outer catheter 1012 of FIG. 2A is positioned against the endocardium 1058 and laser energy is emitted as the inner catheter/fiber/needle assembly 1022 is advanced a first distance, approximately 60% to 80% of the ultrasound estimated thickness of the heart wall, at which point transmission of laser energy ceases, and the injection of a liquid (AGA) 1064 occurs as the inner catheter/fiber/needle assembly 1022 is being withdrawn from the heart wall into the outer catheter 1012. FIG. 7H illustrates the resultant channel and the liquid (AGA) 1064 deposited therein.

If laser energy is allowed to continue as the inner catheter/fiber/needle assembly 1022 is withdrawn, not more than one-half of the first selected distance back into the outer catheter 1012, as shown in FIG. 7I, a wider channel 1078 (inner portion 1080) in the myocardium 1060 is created, due to the double lasing in that area, and is better able to retain the fluid 1064, while still allowing blood from the chamber 1076 to perfuse the heart muscle.

The channels 1078 are typically made about 1 cm apart, as angiogenesis has been shown to extend approximately 0.5 cm from each channel 1078. In addition to either creating all pockets 1062 or channels 1078 in the heart wall, alternating staggered rows of channels 1078 with the needle punctures 1066 and pockets 1062, as shown in FIG. 7H, may be made to obtain the benefits of both better trapping of the liquid (AGA) 1064 in the pockets 1062 and inflow of blood from the chamber 1076 into the heart muscle.

Timing of Administration

Beating Heart

Referring to FIG. 8, when the methods of the present invention are used to treat a beating heart 70, assuming a beating heart rate of 60 beats per minute, it is desired that the above procedures take only about 0.2 to 0.6 seconds, preferably 0.3 to 0.5 seconds, from the time the inner catheter/fiber/needle assembly 22 begins to extend into the heart wall, the pocket 62 or channel 78 is formed, the liquid (AGA) 64 is administered, and the inner catheter/fiber/needle assembly 22 is fully retracted out of the heart wall. If the heart rate is higher than 60 beats per minute, the above times would be proportionally shorter. The above procedure may be conducted over a longer period of time in an arrested or slowed heart 70, for example, during coronary bypass graft surgery, or in a beating heart over a period of several beats, if desired, albeit with a greater risk of an intractable arrhythmia occurring, for example, when a lower powered laser 26 is to be used, which cannot make a channel 78 or pocket 62 in 100 to 300 msec, or if the use of a drug to lower the heart rate is contraindicated. In any case, advancing the device 10 mechanically at a selected rate of speed at a desired energy level enables the channels 78 or pockets 62 to be made with a uniform diameter and depth of coagulation zone surrounding the channel 78 or pocket 62.

It should be noted with regard to all of the embodiments depicted above that the laser 26 can be activated by a foot-pedal 82, finger-button 16, activator rod or by a control unit's 24 sensing the “r” wave of the patient's ECG and supplying an activation signal to the laser 26 or a movable mirror which will divert or enable laser energy to enter into the optical fiber 34. Likewise, the movement of the arm 86 of the advancement mechanism 86 can be activated by a foot-pedal 82, finger-button 16, activator rod or a control system 24 which senses the “r” wave of the patient's ECG.

It is preferred that a control unit 24 monitor the heart 70 by a conventional ECG sensing means operably connected to an ECG device 88 to control the operation of the device 10 by using a signal recognition and timing procedure similar to that disclosed by U.S. Pat. No. 4,788,975, issued to Shturman et al., and incorporated herein by reference.

Preferably, the device 10 enters the heart wall without lasing, forms a pocket 62 within the heart wall or a channel 78 into the heart's chamber 76 by emission of laser energy, injects a therapeutic liquid composition (AGA) 64 and withdraws from the heart wall during diastole, resulting in the pocket 62 shown in FIGS. 6A and 6B. It is desired that the control unit 24 determine when to begin to form the pocket 62 or channel 78 in the heart 70 by interposing an appropriate delay time from the “r” wave of the patient's ECG, taking care to avoid activation of the device 10 during the “t” or “p” wave of the patient's ECG, or in the event of a premature ventricular contraction or any other unusual variation in heart rhythm (arrhythmia).

Forming the pocket 62 or channel 78 and depositing the liquid composition (AGA) 64 when the heart 70 is in diastole is preferred because, at that moment, the electrical activity of the heart 70 is least affected by the trauma of the entry of the needle 32 and the emission of laser energy, reducing the risk of an intractable arrhythmia. Also, the heart chamber 76 is full of blood and the heart wall is at its thinnest.

Shown in FIG. 8, is the timing of an open chest, endoscopic or percutaneous “pocket making” procedure, T1 is the time delay from the “r” wave of the patient's ECG to the inception of movement of the inner catheter/fiber/needle assembly 22 into the heart wall from either the epicardial or endocardial surface. T1 should extend from the “r” wave to the trailing edge of the “t” wave.

T2 is the time during which the inner catheter/fiber/needle assembly 22 is mechanically advanced without lasing the first selected distance into the heart wall, approximately 50 to 100 milliseconds.

T3 is the time during which laser energy is emitted as the inner catheter/fiber/needle assembly 22 is advanced the second selected distance into the heart wall and momentarily stops (laser energy ceases), approximately 50 to 100 milliseconds.

T4 is the time during which, as the inner catheter/fiber/needle assembly 22 withdraws the second selected distance from the heart wall, a therapeutic liquid composition (AGA) 64 is injected, approximately 50 to 100 milliseconds.

T5 is the time during which the inner catheter/fiber/needle assembly 22 withdraws the first selected distance from the heart wall, without lasing, approximately 50 to 100 milliseconds.

In a preferred embodiment, the patient's ECG is displayed on a monitor 90 operably associated with the control unit 24, and a single heart cycle can be displayed thereon. The operator can move Bar 1 by touching a left or right icon on a touch screen or similar device to set the delay time of Bar 1 in relation to the displayed “r” wave of the ECG. Similarly, the operator can move and set Bar 2 by touching a left or right icon on a touchscreen or similar device, setting the total procedure time, T6. Bar 1 should not extend into the “t” wave and Bar 2 should not extend into the “p” wave.

When Bar 1 and Bar 2 have been properly positioned on the patient's ECG, the control unit 24 senses the “r” waves, computes the “r” to “r” heart rate, takes into account the numbers the operator has input for the desired distance of penetration into the heart wall without lasing and the distance of penetration into the heart wall with lasing, and instructs the stepper motor of the fiber advancement withdrawal mechanism 84 to commence its advancement and withdrawal at the proper time at a rate of speed necessary to complete the total travel distance in and out in T6, the time period selected by positioning Bars 1 and 2. In addition, at the proper time, the control unit 24 also signals a shutter mechanism in the control unit 24 or, alternatively, in the laser 26, to open and close at the beginning and end of T2, and the control unit 24 signals the stepper motor of the liquid injection mechanism 28 (or in one preferred embodiment, a syringe injection mechanism 92) to inject the AGA during T4.

Alternatively T1, 2, 3, 4, 5 and 6 can be displayed numerically, and Bars 1 and 2, T1, 2, 3, 4, 5 and 6 can be displayed graphically in distinctive bars or stripes on the display/monitor 90.

If a channel 78 is to be made through the myocardium 60 into the heart chamber 76 from the epicardium 68 in an open chest or endoscopic procedure, T1 remains the time from the “r” wave to the inception of the procedure. T2 is the time from the inception of the procedure through the inner catheter/fiber/needle assembly 22 penetration of the first selected distance into the epicardium 68, without lasing, about 50 to 100 milliseconds. T3 is the time, with emission of laser energy, from T2 until the inner catheter/fiber/needle assembly 22 has traversed the second selected distance through the remainder of the heart wall and has passed into the heart chamber 76 the third desired distance, about 100 to 150 milliseconds. T4 is the time, with lasing, during which the inner catheter/fiber/needle assembly 22 withdraws, for example, T3 plus one-half of T4, about 75 to 125 milliseconds. T5 is the time during which a liquid (AGA) 64 is injected as the inner catheter/fiber/needle assembly 22 withdraws the second one-half of T4, about 50 to 75 milliseconds, and T6 is the time the inner catheter/fiber/needle assembly 22 exits the heart wall, about 50 to 100 milliseconds. T7 is the entire time of the procedure. If no laser energy is to be emitted during the first two withdrawal steps (equal to T3), T4 and T5 are added together.

If a channel 78 is to be created from the heart chamber 76 into the myocardium 60 through the endocardium 58 in a percutaneous procedure, T1 is the time from the “r” wave to the inception of the procedure. T2 is the time the inner catheter/fiber/needle assembly 22 advances, with lasing, the first selected distance into the heart wall, 150 to 200 milliseconds; T3 is the time, with lasing, that the inner catheter/fiber/needle assembly 22 withdraws one-half of the first selected distance (to create a larger diameter space in the myocardium 60 by double lasing) about 75 to 100 milliseconds. T4 is the time, without lasing, that the therapeutic liquid (AGA) 64 is injected as the inner catheter/fiber/needle assembly 22 withdraws the remaining one-half of the first selected distance and into the outer catheter 12, about 75 to 100 milliseconds. T5 is the entire time of the procedure. If no lasing is selected during the withdrawal of the inner catheter/fiber/needle 22, T3 and T4 are additive.

In a procedure where the heart has been arrested, the device 10 may be used with a Holmium:YAG, Excimer, CO₂ or other laser 26 controllably emitting laser energy, preferably pulsed laser energy of a wavelength highly absorbed in water or protein, which creates a steam or gas bubble, whose collapse produces an acoustic shock wave which travels into the tissue, causing the release of endogenous growth factors, which causes angiogenesis to occur, as well as possible increasing cell wall permeability. However, CO₂ lasers cannot be used easily in endoscopic procedures or percutaneous procedures, and excimer lasers are of limited power and generally take 5 or more seconds to make a 4-5 mm pocket 62 in the heart wall (10-15 seconds for a channel). In a procedure where the heart 70 is beating, fiber-optically deliverable laser energy from a laser 26 generating a greater amount of energy, such as a Holmium:YAG laser, is desired.

Before use, the laser 26 is set to deliver a desired amount of energy. The laser 26 is enabled to generate laser energy by depressing a foot-pedal 82 or the like. Activation, insertion, lasing, injection and withdrawal are not critically linked to any specific timing with the heart 70 arrested. However, it is preferred to perform each procedure in the same period of time at the same energy level to assure uniformity of the pockets 62 or channels 78, and within about 0.6 seconds to minimize the coagulation zone, as well as to minimize the time during which the heart 70 is arrested.

As known by those skilled in the art, conventional Holmium lasers have a “ramp-up” time of up to 1 second or longer from the time the laser medium is stimulated to produce laser energy until the time when laser energy is actually provided. Since it is desired that the device 10 be used with any conventional Holmium laser during surgery, an optical fiber 34 can convey laser energy from a laser 26 into a controller, in one embodiment controller 24, which contains an optical coupler and a separate shutter mechanism. The actuator (in one preferred embodiment a foot-pedal 82) of the laser 26 is depressed and laser energy is transmitted to the closed shutter in the controller. When the inner catheter/fiber/needle assembly 22 has advanced to the point where the emission of laser energy is desired to create the pocket 62 or channel 78, the shutter in the controller opens, and laser energy is transmitted through the inner catheter/fiber/needle assembly 22. Alternatively, the laser energy can be diverted into a heat sink by a mirror and, when emission of laser energy is desired, the diverting mirror can move out of the beam path.

Alternatively, the controller 24 can be connected by one or more wires to the laser's CPU (computer processing unit) or the exit shutter mechanism of the laser 26, taking-over its operation. Instead of opening the exit shutter of the laser 26 when the foot-pedal 82 is depressed, the final shutter remains closed and laser energy is emitted into it. When the controller 24 sends a signal to the laser 26 and the exit shutter opens, laser energy is emitted into the optical fiber 34, and the shutter closes at the desired moment. This, however, requires wiring the laser 26, and it may not be practical to wire all types of lasers 26 in the market, and their warranty may be invalidated by doing so.

If laser energy is emitted by a Holmium laser at about 3 Joules per pulse at a repetition rate of about 26 Hertz, for about a 100 millisecond lasing period, approximately 1/10 of 26 or approximately 3 pulses (9 Joules) would be emitted, sufficient to make a channel 78 or pocket 62 approximately 1 mm in diameter and about 2 to 4 mm in length. It should be noted that, the void depends on the size and type of organ or tissue to be treated. Since Holmium laser pulsed energy will create lateral fractures or fissures in the tissue, a void greater than the above described channel 78 or pocket 62 results. Since only about 0.02 cc to 1.5 cc, but preferably about 0.03 cc to about 1.2 cc, of a liquid (AGA) 64 is injected, the space and fissures created would be adequate to hold this volume of mixture.

Ultrasound Guidance

In all of the disclosed devices for practicing the various embodiments of the invention, ultrasound imaging may be used to assist the surgeon in determining the thickness of the heart wall. A conventional ultrasound procedure may be conducted before the procedure, with the physician preparing a chart or remembering from the ultrasound image the thickness of the heart wall at various places, or ultrasound imaging may be performed during the procedure, with the physician or an assistant periodically observing the ultrasound image display and determining the heart wall thickness.

Optionally, an ultrasound emitting and receiving device 55 may be incorporated in the distal end 18 of the outer catheter 12, or in a separate hand held device. The ultrasound image may be displayed on a TV monitor (not shown), so that the surgeon or an assistant can visualize the thickness of the heart wall at the point where the optical fiber 34 is to penetrate the heart wall. In addition, the emission of laser energy into the blood in the heart chamber 76 causes steam bubbles, from the absorption of laser energy, which can be visualized by ultrasound to confirm that the channel 78 into the chamber 76 was completed.

In another embodiment, the aforesaid ultrasound emitter/receiver 55 may also transmit image data to a microcontroller, in one embodiment could be controller 24, which processes the data, calculates and displays the thickness of the heart wall. The microcontroller can also compute and operate the inner catheter/fiber/needle assembly 22 advancement and drug injection mechanisms 28, such that the inner catheter/fiber/needle assembly 22 is advanced, the pocket 62 or channel 78 is created by the emission of laser energy, the therapeutic liquid (AGA) 64 is injected and the inner catheter/fiber/needle assembly 22 is withdrawn the desired distances, based on pre-selected instructions.

Furthermore, with regard to all of the devices described, as the inner catheter/fiber/needle assembly 22 is advanced into the tissue, organ or heart wall while the laser 26, is firing, a plasma of hot gasses from the vaporization of tissue forms ahead of the inner catheter/fiber/needle assembly 22. These hot gasses cannot escape backwards, as the tissue hugs the needle 32 in the channel 78, and solid tissue remains ahead of inner catheter/fiber/needle assembly 22. These hot gasses accumulate and cause the diameter of the channel 78 to increase as the inner catheter/fiber/needle assembly 22 advances through the tissue, which may result in a larger ultimate pocket 62 or channel 78 in the tissue, organ or heart wall. However, to limit the zone of coagulation about the channel 78 and lateral damage to the myocardium 60, it may be necessary to advance the inner catheter/fiber/needle assembly 22 at a relatively fast rate for a very short time at a given energy level to achieve a desirable and uniform channel or pocket size and coagulation zone.

Laser Source

Laser sources suitable for adaptation to the methods of the present invention, and use of the device 10 of the present invention are described in the art. In a preferred embodiment, the laser device 26 produces energy from a Holmium:YAG laser or comparable laser at a wavelength of 1400 to 2200 micrometers. Energy from an excimer laser (300 to 400 micrometers), Argon laser (488-520 micrometers), KTP laser (532 micrometers), Nd:YAG laser (1064 micrometers), Erbium laser (2940 micrometers) or any other source of laser energy which is able to be transmitted through optical fibers, or in a pulsed, gated, or continuous wave may be utilized. Preferably, a multi-head Holmium laser, as described in U.S. Pat. No. 5,242,438 to Saadatmanesh et al., is preferred.

Needle/Tip-Optical Fiber/Inner Catheter Assembly

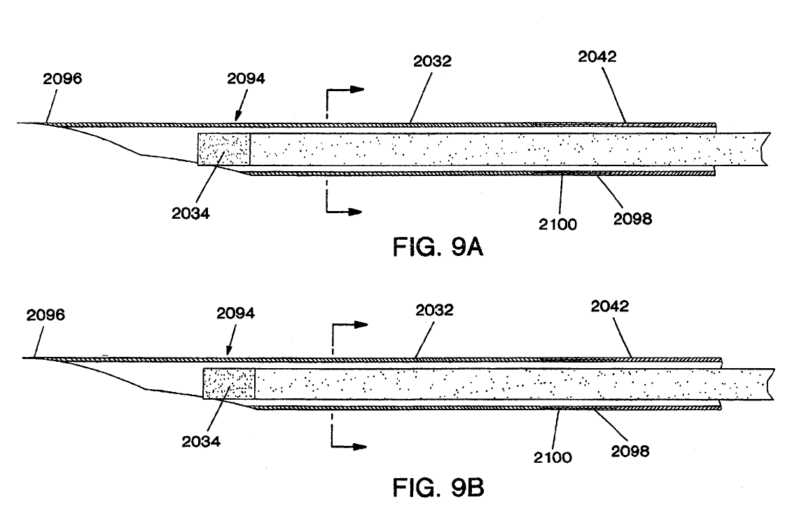

In one embodiment, a simple device 2010 for penetrating tissue mechanically using a syringe needle 2032 in which an optical fiber 2034 is encased, for the application of laser energy after the device 2010 has first penetrated a selected distance into the tissue has been designed.

Where appropriate, the last three digits of the 2000 series of numerals depicted in FIGS. 9A-B are connected to elements which have the same function and/or structure as those depicted with regards to FIGS. 1-8.

Since the length of the needle 2032 must sometimes be limited, when for example, the fiber 2034 must be bent at a sharp angle to pass through a canula or to articulate in an outer catheter 2012 in a desired direction in a confined space, for example in the left ventricle of the heart, the needle 2032 must be firmly anchored to the optical fiber 2034. Otherwise, the needle 2032 will not advance in synchrony with the optical fiber 2034, or the needle 2032 can become detached.

The device 2010 of the invention solves this problem by crimping the needle 2032 to the optical fiber 2034 at about the 9 and 3 O'Clock positions (when viewed in cross-section), resulting in an oval shape with fluid conveying channels 2044 at about the 12 and 6 O'Clock positions, as shown in FIG. 2C.

The inner catheter/fiber/needle assembly 2022 will comprise, in one embodiment, an optical fiber 2034 extending through the inner catheter 2042 where about 6 to 15 mm of the distal end 2094 of the optical fiber 2034 is encased within an appropriate length of syringe needle 2032 (preferably about 8 to 17 mm), preferably with a sharp or double-beveled distal end 2096.

As illustrated in FIG. 9A, a thin-walled inner catheter 2042 is disposed about the optical fiber 2034, from whose distal end 2094 the buffet coat has been removed. The inner catheter 2042 is affixed over a proximal end 2098 of the needle 2032, by an adhesive or the like, so that fluid communication through the needle 2032 is obtained, without the catheter 2042 being thick or stiff, so the motive force can be applied solely to the optical fiber 2034.

As illustrated in FIG. 9B, a symmetrical outer surface of the inner catheter 2042 to needle 2032 junction can be achieved by creating a flange 2100 at the proximal end 2098 of the needle 2032, over which the inner catheter 2042 can be attached by adhesive or the like. Apparatus similar to this design are relatively simple to manufacture at reasonable cost, and are relatively durable in use. A sample embodiment of such a device has been used to make more than 400 channels in bovine heart tissue with emission of laser energy during in vitro testing.

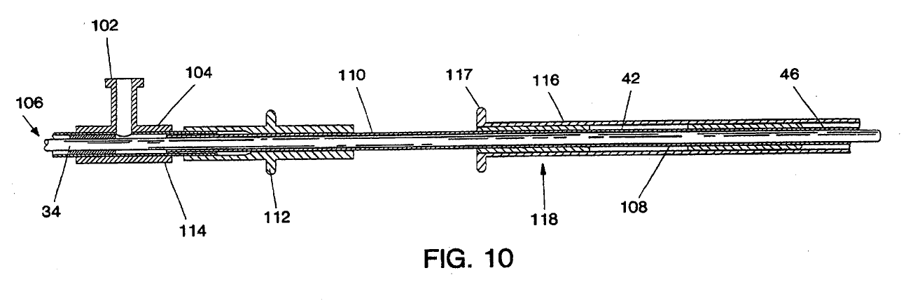

As illustrated in FIG. 10, a luer lock 102 forming a port of a “y” or “t” connector 104, which is attached by adhesive to the optical fiber 34 at a point about 25 to 60 cm from the proximal end 106 of the optical fiber 34 can be used for infusion of the liquid (AGA) 64 into the space between the optical fiber 34 and an inner catheter 42 attached by an adhesive to the distal end 108 of an inner metal sleeve 110 with a flange 112, which is attached to the distal end 114 of the “y” or “t” connector 104. The distal end of 108 the inner metal sleeve 110 is movably disposed within an outer sleeve 116 (metal or other suitable material) shown with a flange 117 attached by adhesive to the proximal end 118 of the outer catheter 12.

In devices for practicing the methods of the invention, the apparatus will have a source of pulsed laser energy optically connected to the proximal end 106 (opposite from the needle end) of an optical fiber 34 for delivery of laser energy to the inner catheter/fiber/needle assembly 22. In a preferred embodiment, the optical fiber 34 extends into the needle 32 from within the lumen 46 of an inner catheter 42 which is in fluid communication with a source of an AGA 64 in a liquid suspension.

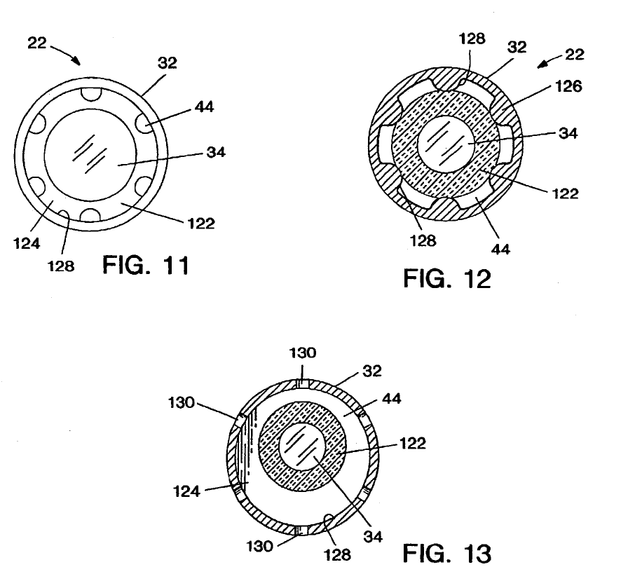

As shown in FIGS. 11-13, three additional embodiments of the inner catheter/fiber/needle assembly 22 of a device 10 for practicing the invention are depicted. While described in terms of a needle 32, it is also contemplated, as discussed above, that a pointed, tapered or blunt ended tip 36 may also be suitably formed for making the mechanical puncture of the heart muscle, and thus may incorporate the features described herein with reference to a needle 32. Such a tip 36 may be formed from suitable metal or plastic.

As shown in FIG. 11, fluid channels 44 through the needle 32 are cut within a buffer or jacket 122 which encases the optical fiber 34 and within the bore 124 of the needle 32 allowing fluid communication therethrough to the lumen 46 of the inner catheter 42.

FIG. 12 depicts an embodiment similar to that of FIG. 11, however, protrusions or flanges 126 from the inner surface 128 of the bore 124 of the needle 32 crimp down upon the fiber optic jacket 122, holding the optical fiber 34 firmly within and in place, fluid channels 44 being available in the spaces between the protrusions or flanges 126.

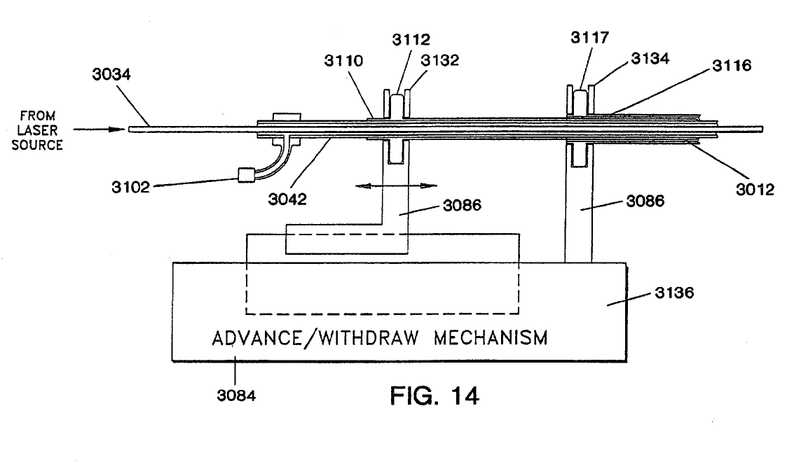

As shown in FIG. 13, instead of fluid exiting from the distal end 96 of the needle 32, which can also be affixed to the distal end 94 of optical fiber 34 with adhesive, one or more side ports 130 proximal to the distal end 96 of the needle 32 are provided to allow fluid 30 or liquid (AGA) 64 to exit.

Where appropriate, the last three digits of the 3000-10000 series of numerals depicted in FIGS. 14-25 are connected to elements which have the same function and/or structure as those depicted with regards to FIGS. 1-9.

As seen in FIG. 14, for attaching the fiber 3034/inner catheter 3042 to the advancement/withdrawal mechanism 3084, an inner metal sleeve 3110 with a flange 3112 can be disposed over and attached by an adhesive to a “y” or “t” connector 3104 affixed to the optical fiber 3034 about ¼ to ⅓ of the length of the fiber 3034 from the source of laser energy (not shown). The distal end of the inner metal sleeve 3110 is movably disposed within an outer sleeve 3116 with a flange 3117, which is attached to the proximal end 3118 of the outer catheter 3012. The first metal sleeve 3110 is removably attached to a first clamp 3132 attached to the arm 3086 of the advancement/withdrawal mechanism 3084. The second sleeve 3116 is removably attached to a second clamp 3134 attached to the frame or housing 3136 of the advancement/withdrawal mechanism 3084.

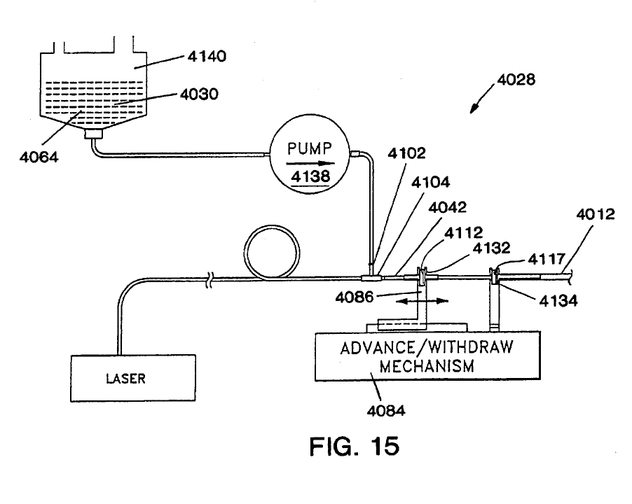

In FIG. 15, an alternative embodiment of the device 4010 of the invention is shown. In this embodiment, the liquid injection mechanism 4028 comprises a pump 4138 and reservoir 4140 in fluid communication through the inner catheter 4042.

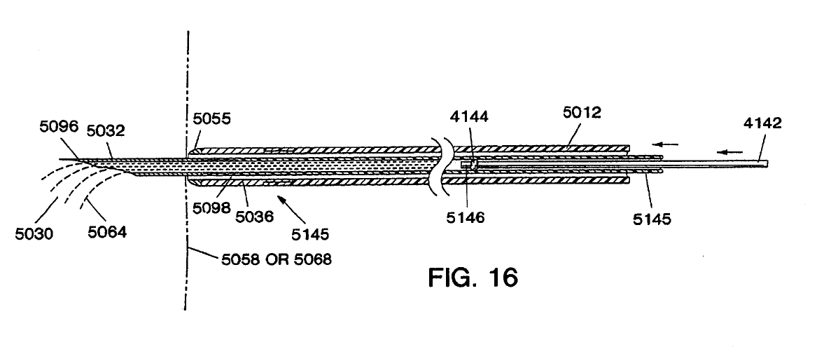

FIG. 16 illustrates an embodiment of the invention which does not utilize laser energy. Using a moveable obturator 5142 (optical fiber, hypo tubing with the distal end sealed, a plastic rod or other material) with an “O” ring 5144 affixed near its distal end 5146, this moveable inner catheter/needle assembly, referred to generally as 5145, is advanced a selected distance into the tissue, organ or heart wall manually or by a first advancement means (not shown). The obturator 5142 is then advanced manually by a second advancement means (not shown) a selected distance to displace a desired amount of the therapeutic liquid (AGA) 5064 from the inner catheter 5042, against which the “O” ring 5144 forms a seal. The outer catheter 5012 may, optionally, contain an ultrasound transducer 5055, so the thickness of the heart wall at the point of contact may be ascertained by the operator.

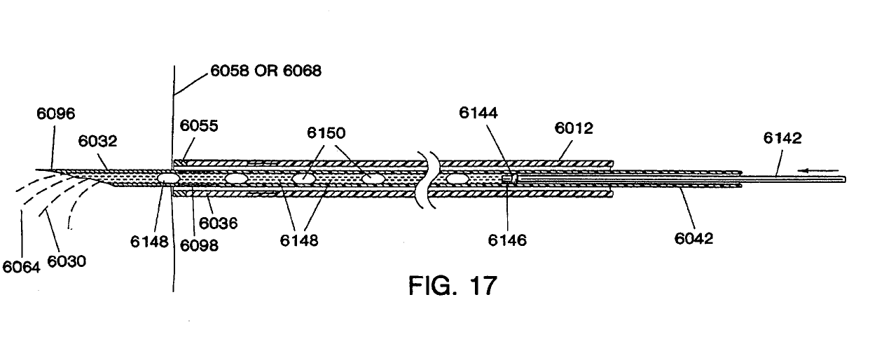

As seen in FIG. 17, the fluid channel of the device 6010 of FIG. 16 contains aliquots 6148 of the therapeutic liquid (AGA) 6064 separated by bubbles 6150 of a gas (air, CO₂, nitrogen or the like). The obturator 6142, with an “O” ring 6144 to form a seal, is manually or mechanically advanced a distance sufficient to eject one or more aliquots 6148 of a therapeutic liquid (AGA) 6064 into the tissue, organ or heart wall.

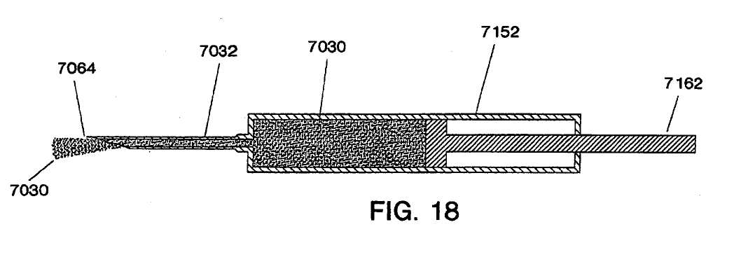

Another embodiment of the invention including the liquid injector mechanism 7028, again without using laser energy, is shown in FIG. 18. In this embodiment the needle 7032 of a conventional syringe 7152 is advanced a desired distance into a tissue, organ or heart wall, manually or by a first advancement means (not shown). The plunger 7163 of the syringe 7152 is advanced manually or by a second advancement means (not shown) a selected distance to expel a desired quantity of the therapeutic liquid (AGA) 7064 into the tissue, organ or heart wall.

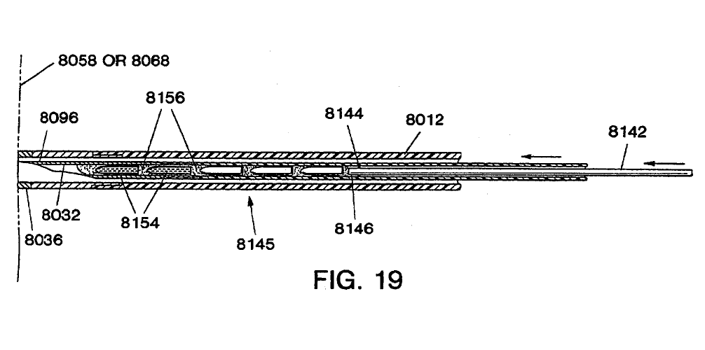

As seen in FIG. 19, another embodiment of the invention is shown. One or more capsules or pellets 8154 of bone marrow extract, after screening, centrifuging and removal of fat, optionally mixed with a vehicle such as cocoa butter or a polyethylene glycol (i.e., CARBOWAX®) among others which melt at body temperature, and pelletizing, optionally in a pointed end or bullet shaped configuration, are lined up within the inner catheter 8042 of FIG. 16, separated by liquid or preferably by a gelation 8156. The inner catheter/needle assembly 8145 is advanced a selected distance into the tissue, organ or heart wall manually or by a first advancement mechanism (not shown). The obturator 8142 is then advanced manually or by a second advancement mechanism (not shown) a distance necessary to expel one or more of a desired number of the pellets 8154, which may optionally be enriched with stem cells collected from the patient's peripheral blood, and which may include adhesion molecules to enhance the attachment of the liquid (AGA) 8064 in the desired tissue, organ or heart wall.

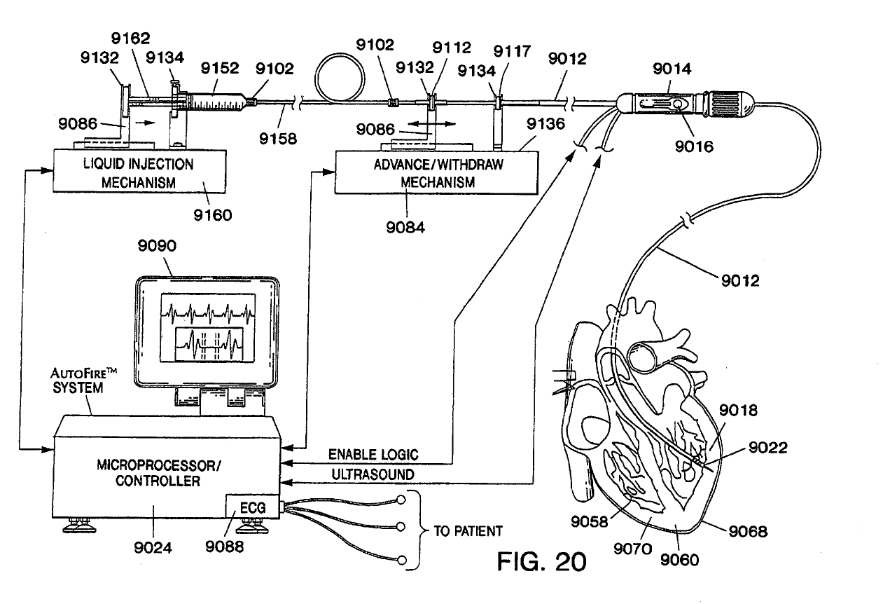

As seen in FIG. 20, in an alternate embodiment of the present invention in which laser energy is not used (and no optical fiber is included), the inner catheter 9042 is connected at its proximal end 9158 to a short length of syringe needle 9032, maintaining a fluid channel with the syringe 9152 as described heretofore. The inner catheter 9042 is movably disposed within an outer catheter 9012, whose directable (steerable) distal end 9018 can he positioned at the inner surface of the left ventricle of the heart or upon an organ or other tissue. When the activation button 9016 on the handpiece 9014 is pressed, a microprocessor 9024, based on pre-selected parameters, causes an advance/withdrawal mechanism 9084 to advance and withdraw the inner catheter/needle assembly 9145; and a liquid injector mechanism 9028 to advance the plunger 9162 of the syringe 9152 to accomplish the desired result. Optionally, this process may be synchronized from the “r” wave of the patient's ECG.

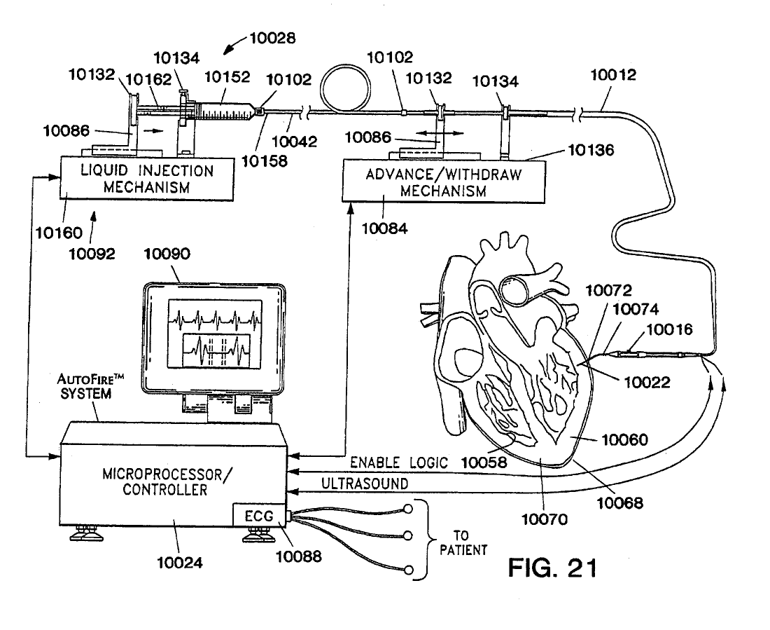

As seen in FIG. 21, the device of FIG. 20 is illustrated, except that the outer catheter 10012 terminates in a metal cannula extending from a handpiece 10014, which can be positioned on the exterior surface or of the heart 10070, an organ or other tissue. In all other respects, this embodiment is similar to that shown in FIG. 20.

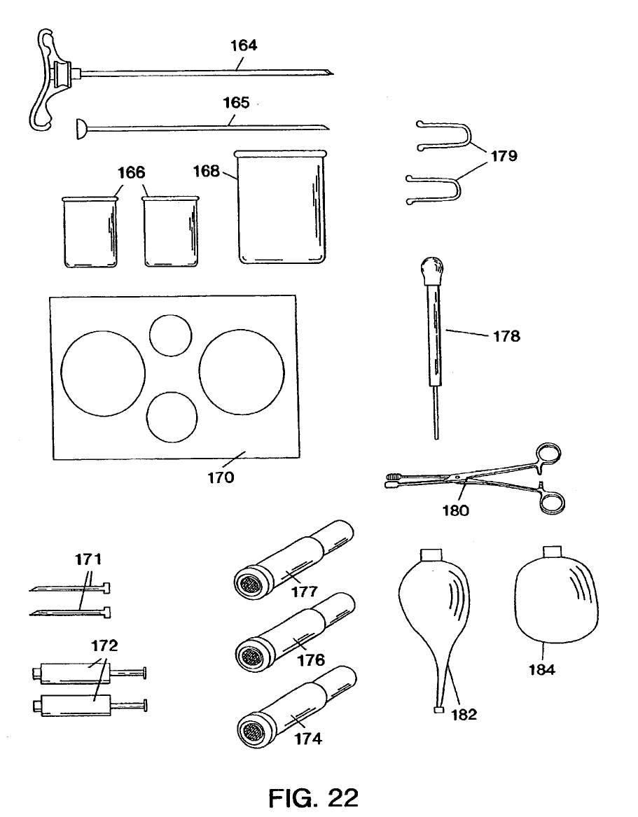

As seen in FIG. 22, an example of a kit for removal of bone marrow and preparation of bone marrow for injection is shown. The kit, in addition to other components (not shown) may minimum contain the following:

1 Steis, Wolf, Rosenthal or other aspiration Needle 164 with stylus 165, 11 to 14 gauge, 4½ to 5½ long needle.

2 250 ml beakers 166 (optional).

1 500 ml beaker 168 (optional).

1 Beaker holder 170 (optional).

2 2 needles 171 (10-14 gauge).

2 20 cc syringes 172 (with bored out lumens).

3 Cut-off 20 cc syringes with wide plungers and screens (1 with 0.201 mm width screen 174, 1 with 0.307 mm width screens 176, and 1 with 0.4 mm wide screen 177).

1 Pipette 178.

2 Centrifuge tubes (179).

1 Hemostat 180.

1 Scissors.

2 EDTA bottles (optional) (not shown).

2 Processing bags 182 with anticoagulant.

1 Storage bag 184.

The procedure is performed under sterile conditions and under general anesthesia in an operating room. A solution of about 4 ml of heparin, without preservatives (Connaught Laboratories) in 100 ml of serum free tissue culture medium (Stem Pro-34™ SFM or Knockout™SR, Life Technologies, Rockville, Md.) is prepared. The beakers and other utensils are rinsed with the mixture.

The procedure is performed as described by Thomas, et al., in “Technique for Human Marrow Grafting”, Blood 36(4); pg. 507-515 (October, 1970), by Deeg, H J et al. in “A Guide to Bone Marrow Transplantation”, 2nd Ed., Springer-Verlag; Pg. 89-94 (1992), or by other means known in the art.

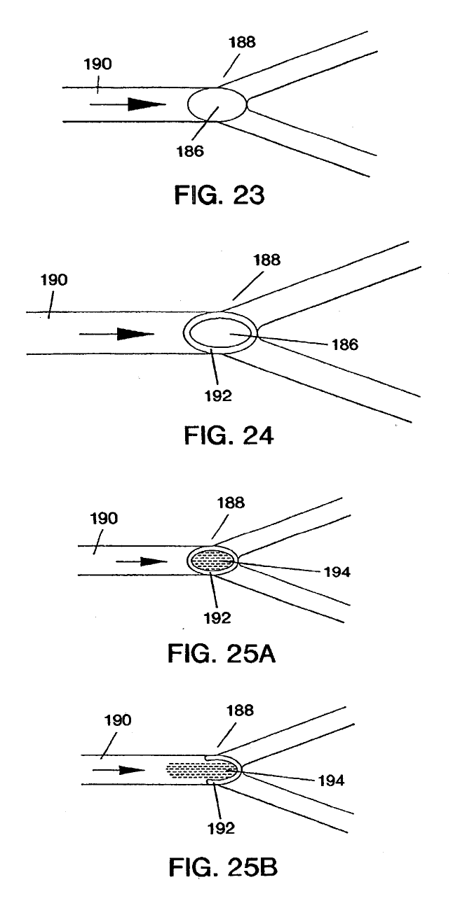

FIG. 23 shows a human stem cell 186, whose diameter is approximately 8.2+/−1.1 microns to 8.7+/−1.2 microns, according to Gao, D. Y. et al., “Fundamental Cryobiology of Human Hematopoietic Progenitor Cells. I:Osmatic Characteristics and Volume Distribution”, Cryobiology 36(1):40-48 (February 1998), lodged at the bifurcation 188 of a larger blood vessel 190. The arrow indicates the direction of blood flow.

FIG. 24 shows a human stem cell 186 encapsulated in a liposome 192, with a diameter of about 10 to 15 microns, lodged at the bifurcation 188 of a blood vessel 190.

FIG. 25A shows intercellular bone marrow 194 material encapsulated in a liposome 192, with a diameter of about 7-10 microns, lodged at the bifurcation 188 of a blood vessel 190.

FIG. 25B shows the partially decomposed liposome 192 encapsulating the intercellular bone marrow 194 material of FIG. 24A partially decomposed, with the decomposition and release of material 194 into the blood vessel occurring at the flow pressurized face of the liposome 192.

A study was conducted at the Texas Heart Institute of St. Luke's Hospital in Houston to compare the injection of autologous bone marrow into the heart muscle of pigs during diastole with the injection into the heart muscle of pigs of the same volume of saline.

Ten Yorkshire pigs were used in the study. The pigs were each assigned a number and tagged. Five of the pigs were randomized into each of two groups (Group A—injection of autologous bone marrow into the heart wall during diastole and Group B—injection of the same volume of saline into the heart wall during diastole).

All of the pigs underwent open chest surgery and ultrasound imaging to provide a Base Line heart wall motion assessment, as known in the art. An Ameroid constrictor was then implanted about their circumflex coronary artery. The Ameroid constrictor slowly closes the artery, simulating the closure of an artery by the growth or rupture of a plaque deposit. Approximately 5 weeks after implantation of the Ameroid constrictor, all of the pigs underwent a second open chest surgery and an ultrasound imaging to assess their heart wall motion. The pigs in both Groups A and B had significantly impaired heart wall motion, confirming that blood flow to the area of the heart supplied by the circumflex artery was significantly reduced.

A small amount of bone marrow was aspirated (withdrawn) by syringe from the sternum (breast bone) of the pigs. The five pigs in Group A received 15 injections of 0.1 cc each of autologous bone marrow into the heart wall in the area of the heart supplied by the circumflex artery during diastole. The five pigs in Group B had the same volume of saline injected into the heart wall in the area of the heart supplied by the circumflex artery during diastole. The same size needle was used to inject the bone marrow or saline.

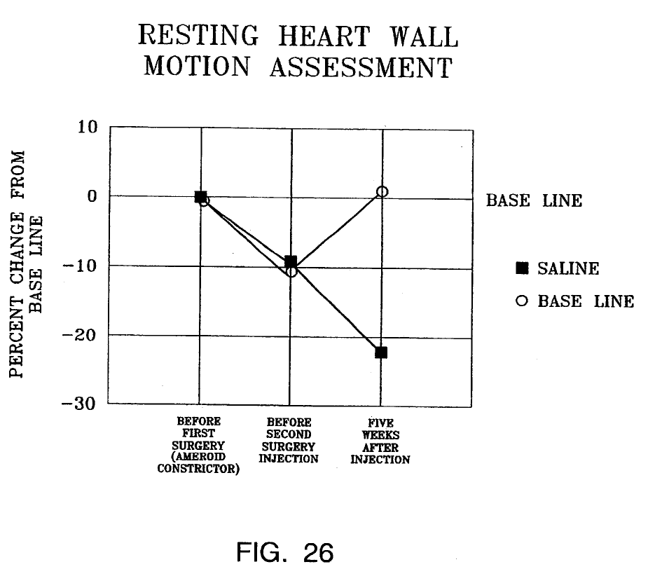

Approximately 5 weeks after the second surgery, all of the pigs underwent a third open chest surgery and ultrasound imaging to assess their heart wall motion. One of the pigs in Group A suffered from ventrictilar fibrillation and could not be assessed. One pig in Group B died following the second open heart surgery. The average heart wall motion assessment of the four pigs in Group A and the four pigs in Group B by independent physicians who were blinded (unaware) as to which Group the numbered pigs were assigned is shown in FIG. 26.

As can be seen, at the time of the second surgery, the heart wall motion assessment of the pigs in both Groups A and B declined significantly from the Base Line assessment at the time of the first surgery. At the time of the third surgery, however, the pigs in Group A, which received injections of autologous bone marrow, had a significantly improved heart wall motion assessment, slightly surpassing their initial Base Line assessment. However, the heart wall motion assessment of the pigs in Group B, which received injections of saline, continued to decline. It should be noted that the Ameroid constrictor was still in place in all of the pigs, restricting blood flow to the heart wall area supplied by the circumflex artery.

The foregoing data demonstrates that autologous adult bone marrow is effective in the repair of cardiac tissue and creation of blood vessels.

While particular embodiments of the present invention has been shown and described, it will be appreciated by those skilled in the art that changes and modifications may be made hereto without departing from the invention in its broadest aspects and as set forth in the following claims.

I claim:

1. A method for repairing tissue in vivo and comprising:
providing a physiologically compatible bone marrow composition that includes bone marrow and at least one additional autologous growth agent in phosphate buffered saline; and
introducing a growth enhancing amount of said physiologically compatible bone marrow composition within tissue to be repaired.

2. The method in accordance with claim 1 wherein said introduced bone marrow composition is enriched with stem cells selected from the group consisting of embryonic stem cells and hematopoietic stem cells.

3. The method in accordance with claim 1 wherein said introduced bone marrow composition includes adhesion molecules.

4. The method in accordance with claim 1 wherein said introduced bone marrow composition is encapsulated in liposomes having a diameter of at least about 7 microns.

5. The method in accordance with claim 1 wherein said bone marrow composition is introduced into a cavity created using light energy transmitted by an optical fiber.

6. The method in accordance with claim 1 wherein said bone marrow composition is thixotropic.

7. The method in accordance with claim 1 wherein said bone marrow composition includes a hydrocolloid.

8. A method for repairing bone tissue in vivo and comprising:
providing a physiologically compatible bone marrow composition that includes bone marrow and at least one additional autologous growth agent in phosphate buffered saline; and
introducing a growth enhancing amount of said physiologically compatible bone marrow composition within the bone tissue to be repaired.

9. The method in accordance with claim 1 wherein said physiologically compatible bone marrow is provided in pellet form.

10. A method for repairing tissue in vivo and comprising:
providing a physiologically compatible bone marrow composition wherein said bone marrow composition includes bone marrow and at least one autologous growth agent;
creating a cavity within defective tissue in vivo; and
depositing said physiologically compatible bone marrow composition in pellet form into said created cavity.

11. A method for revascularizing cardiac tissue in vivo and comprising:
providing a physiologically compatible composition which composition includes bone marrow; and
introducing said physiologically compatible composition into said cardiac tissue.

12. The method in accordance with claim 11 wherein said composition comprises at least one additional autologous growth agent.

13. The method in accordance with claim 11 wherein said composition is enriched with stem cells selected from the group consisting of embryonic stem cells and hematopoietic stem cells.

14. The method in accordance with claim 11 wherein said composition includes adhesion molecules.

15. The method in accordance with claim 11 wherein said composition is encapsulated in liposomes having a diameter of at least about 7 microns.

16. The method in accordance with claim 11 wherein said cavity is created using light energy transmitted by an optical fiber.

17. The method in accordance with claim 11 wherein said composition is thixotropic.

18. A composition for repairing tissue in vivo and comprising:
a supply of bone marrow physiologically compatible with said tissue and enriched with autologous stem cells.

19. The composition in accordance with claim 18 in phosphate buffered saline.
